(19) United States
(12) Patent Application Publication
(10) Pub. No.: US 2009/0169620 A1
(43) Pub. Date: Jul. 2, 2009

(54) ORALLY DISINTEGRATING TABLET COMPOSITIONS OF TEMAZEPAM

(76) Inventors: Gopi M. VENKATESH, Vandalia, OH (US); James M. Clevenger, Vandalia, OH (US); Jin-Wang Lai, Springborro, OH (US); Vivek Purohit, East Lyme, CT (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/339,908

(22) Filed: Dec. 19, 2008

Related U.S. Application Data
(60) Provisional application No. 61/015,931, filed on Dec. 21, 2007.

(51) Int. Cl. A61K 9/20; A61K 31/5513

(52) U.S. Cl. 424/464; 514/221

(57) ABSTRACT

The compositions of the present invention are orally disintegrating tablet compositions comprising a therapeutically effective amount of at least one drug such as temazepam, 0.5-3% of an ODT binder polymer, a sugar alcohol and/or saccharide, and a disintegrant.

### CROSS REFERENCE TO RELATED APPLICATIONS

This application claims priority to U.S. Provisional Appl. No. 61/015,931, filed Dec. 21, 2007, which is herein incorporated by reference in its entirety for all purposes.

### TECHNICAL FIELD

The present invention relates to orally disintegrating tablet compositions, and improved methods of making such compositions. The compositions of the present invention comprise a drug, 0.5-3% of an ODT binder polymer, a sugar alcohol and/or saccharide, and a disintegrant.

### BACKGROUND OF THE INVENTION

Dysphagia, or difficulty in swallowing due to fear of choking, is common among all age groups. For example, it is observed in about 35% of the general population, as well as an additional 30-40% of elderly institutionalized patients and 18-22% of all persons in long-term care facilities, many of whom are required to consume medications on a regular basis to maintain their quality of life. This leads to poor compliance or even non-compliance with the treatment and thus has a negative impact on the efficacy of the treatment. Conventional capsule or tablet dosage forms are also inconvenient for the “people on the move” who often do not have access to drinking water or fluids. In addition, some pediatric, geriatric, and psychiatric patient populations exhibit “cheeking” behavior (i.e., holding the oral dosage form in the cheek) to avoid swallowing the medication. Accordingly, ODT formulation would be desirable to improve patient compliance, particularly among elderly and institutionalized patients, because ODT formulations are easier to swallow and prevent “cheeking”.

However, in order to be pharmacologically acceptable, ODT formulations must be palatable, e.g. have acceptable organoleptic properties such as good taste and mouthfeel, because ODT tablets are designed to disintegrate in the mouth of the patient. For bitter tasting drugs, the ODT formulation may require a taste-masking polymer to improve the taste characteristics of the formulation, and to provide a “creamy” mouthfeel. In addition, the ODT formulation must also provide acceptable pharmacokinetics and bioavailability to provide the desired therapeutic effect. These desired properties of an ODT formulation can be contradictory, in that taste-masking can inhibit or delay drug release, thereby providing unacceptable pharmacokinetic properties. Conversely, components of the formulation that promote rapid release may result in undesirable taste or mouthfeel properties. Finally, any ODT composition with suitable organoleptic and pharmacokinetic properties must also be manufactured at commercially useful rates and yields.

Accordingly, an acceptable ODT formulation must balance these contradictory characteristics in order to provide a palatable (e.g., taste-masked), fast disintegrating composition with acceptable pharmacokinetics.

### SUMMARY OF THE INVENTION

The present invention is directed to orally disintegrating tablet compositions comprising a therapeutically effective amount of at least one drug (e.g., temazepam), 0.5-3% of an ODT binder polymer, a sugar alcohol and/or saccharide, and a disintegrant.

In another embodiment, the orally disintegrating tablet compositions of the present invention comprising drug particles and rapidly-dispersing microgranules, wherein the rapidly-dispersing microgranules comprise the sugar alcohol and/or saccharide in combination with the disintegrant, and the drug particles comprise the drug and an ODT binder polymer.

In another embodiment, the orally disintegrating tablet compositions of the present invention comprise drug-coated beads and rapidly-dispersing microgranules, wherein the rapidly-dispersing microgranules comprise the sugar alcohol and/or saccharide in combination with the disintegrant, and the drug-coated beads comprise inert cores coated with at least one drug and an ODT binder polymer.

In another embodiment, the present invention is directed to a method of preparing an orally disintegrating tablet composition comprising mixing at least one drug, 0.5-3% of the ODT binder polymer, a sugar alcohol and/or saccharide, and the disintegrant, and compressing said mixture, thereby forming an orally disintegrating tablet.

In another embodiment, the method of the present invention comprises granulating a mixture comprising at least one drug and the ODT binder polymer, thereby forming drug microgranules; granulating a disintegrant mixture comprising a sugar alcohol and/or saccharide in combination with a disintegrant in the presence of an ODT binder, thereby forming rapidly-dispersing microgranules; blending the drug microgranules and rapidly-dispersing microgranules; and compressing the blend of drug microgranules, rapidly-dispersing microparticles, and optionally other pharmaceutically acceptable excipients (e.g., a flavorant, a sweetener, a compression aid, etc.), thereby forming an orally disintegrating tablet.

In another embodiment, the method of the present invention comprises dissolving or suspending at least one drug and a drug-layering binder polymer in a solvent; coating the solution or suspension of the drug onto an inert core to form drug-layered beads; granulating a disintegrant mixture comprising a sugar alcohol and/or saccharide in combination with a disintegrant, thereby forming rapidly-dispersing microgranules, and compressing the blend of drug-layered beads, rapidly-dispersing microparticles, and optionally other pharmaceutically acceptable excipients (e.g., a flavorant, a sweetener, a compression aid, etc.), thereby forming an ODT.

In another embodiment, the present invention is directed to a method of improving patient compliance with the administration of a drug such as temazepam, comprising administering an orally disintegrating tablet composition comprising a therapeutically effective amount of a drug (e.g., temazepam), 0.5-3% of an ODT binder polymer, a sugar alcohol and/or saccharide, and a disintegrant to a patient in need thereof.

### BRIEF DESCRIPTION OF THE DRAWINGS

FIG. 1 shows the blend uniformity sampling locations for a 10 ft³ V-blender, and corresponding assay values determined using a validated analytical methodology.

FIG. 2A shows the tablet weight variations for the 7.5 mg temazepam ODT dosage forms of Example 3.

FIG. 2B shows the tablet weight variations for the 30 mg temazepam ODT dosage forms of Example 3.

FIG. 3 shows multimedia dissolution profiles for 30 mg temazepam ODT dosage forms vs. 30 mg Restoril® capsules, USP official media, pH 4.5 (top); 0.1N HCl, pH 6.8 (middle) and 30 mg, 22.5 mg, 15 mg & 7.5 mg doses of ODTs or Restoril® Capsules in the USP official media (bottom).

FIG. 4A compares the mean plasma concentration-time profiles for 30 mg temazepam ODT, dosed without water (A), or with water (B) vs. 30 mg Restoril® capsules in normal human subjects under fasting conditions.

FIG. 4B compares the mean AUC (with standard deviations) for 30 mg temazepam ODT, dosed without water (A), or with water (B) vs. 30 mg Restoril® capsules in normal human subjects under fasting conditions.

FIG. 4C compares the mean Cₘₐₓ (with standard deviations) for 30 mg temazepam ODT, dosed without water (A), or with water (B) vs. 30 mg Restoril® capsules in normal human subjects under fasting conditions.

FIG. 5 compares the mean plasma concentration-time profiles for 30 mg temazepam ODT vs. 30 mg Restoril® capsules in normal human subjects under fed conditions.

### DETAILED DESCRIPTION OF THE INVENTION

All applications, patents and other documents cited herein are incorporated by reference in their entirety for all purposes.

The present invention is directed to drug-containing ODT compositions, and improved methods of preparing such compositions as described herein. The ODT compositions of the present invention have good organoleptic properties, yet retain drug release properties which are bioequivalent to, or superior to (i.e., faster than) conventional oral dosage forms. In addition, the ODT compositions of the present invention exhibit sufficient hardness and sufficiently low friability to permit packaging in HDPE bottles, and push-through film backed or peel-off paper backed blister packs, using conventional equipment for storage, transportation and commercial distribution.

The terms “orally disintegrating tablet”, “orally dispersible tablet”, “fast dissolving tablet”, or “ODT” refer to a solid dosage form which disintegrates rapidly in the oral cavity of a patient after administration. The term “substantially disintegrates” means a disintegration of the tablet largely into constituent particles which were previously compressed into monolithic tablets.

The term “substantially dissolves” means the percentage of the drug released or dissolved, i.e., at least about 50%, at least about 60%, at least about 70%, at least about 80%, at least about 90%, or about 100% of drug dissolved or released from the ODT composition.

The term “temazepam” includes prodrugs, salts, solvates, and/or esters of temazepam (7-chloro-1,3-dihydro-3-hydroxy-1-methyl-5-phenyl-2H-1,4-benzodiazepin-2-one).

The term “unit dose” refers to a pharmaceutical composition containing an amount of drug intended to be administered to a patient in a single dose.

The term “drug”, “active” or “active pharmaceutical ingredient” as used herein includes a pharmaceutically acceptable and therapeutically effective compound suitable for treating diseases, symptoms of diseases, and medical conditions, as well as pharmaceutically acceptable salts, stereoisomers and mixtures of stereoisomers, solvates (including hydrates), and/or esters thereof.

Non-limiting examples of classes of suitable active pharmaceutical ingredients include, but are not limited to benzodiazepines, analgesics, antihypertensives, antianxiety agents, anticlotting agents, anticonvulsants, anti-diabetic agents, blood glucose-lowering agents, decongestants, antihistamines, anti-inflammatory agents, antitussives, antineoplastics, beta blockers, anti-rheumatic agents, anti-inflammatories, antipsychotic agents, cognitive enhancers, anti-atherosclerotic agents, antiobesity agents, anti-impotence agents, anti-infective agents, anti-infective agents, hypnotic agents, anti-Parkinsonism agents, anti-Alzheimer's disease agents, anti-depressants, and antiviral agents, glycogen phosphorylase inhibitors, cholesterol ester transfer protein inhibitors, CNS (central nervous system) stimulants, dopamine receptor agonists, anti-emetics, gastrointestinal agents, psychotherapeutic agents, opioid agonists, opioid antagonists, anti-epileptic drugs, histamine H₂ antagonists, anti-asthmatic agents, smooth muscle relaxants, and skeletal muscle relaxants. In a particular embodiment, the active pharmaceutical ingredients suitable for use in the ODT compositions of the present invention include drugs that are sensitive to processing conditions (e.g., thermally sensitive, have a low explosive threshold, shear sensitive, or otherwise have a tendency to degrade during processing).

Specific examples of benzodiazepines include alprazolam, bromezepam, diazepam, lorezepam, clonezepam, temazepam, oxazepam, flunitrazepam, triazolam, chlordiazepoxide, flurazepam, estazolam, lormetazepam, midazolam, nitrazepam, and mexazolam; analgesics include acetaminophen, rofecoxib, celecoxib, morphine, codeine, oxycodone, hydrocodone, diamorphine, pethidine, tramadol, buprenorphine; antihypertensives include prazosin, nifedipine, lercanidipine, amlodipine besylate, trimazosin and doxazosin; specific examples of antianxiety agents include hydroxyzine hydrochloride, lorazepam, buspirone hydrochloride, pazepam, chlordiazepoxide, meprobamate, oxazepam, trifluoperazine hydrochloride, clorazepate dipotassium, diazepam; specific examples of anticlotting agents include abciximab, eptifibatide, tirofiban, lamifiban, clopidogrel, ticlopidine, dicumarol, heparin, and warfarin; specific examples of anticonvulsants include phenobarbital, methylphenobarbital, clobazam, clonazepam, clorezepate, diazepam, midazolam, lorazepam, felbamate, carbamezepine, oxcarbezepine, vigabatrin, progabide, tiagabine, topiramate, gabapentin, pregabalin, ethotoin, phenyloin, mephenyloin, fosphenyloin, paramethadione, trimethadione, ethadione, beclamide, primidone, brivaracetam, levetiracetam, seletracetam, ethosuximide, phensuximide, mesuximide, acetazolamide, sulthiame, methazolamide, zonisamide, lamotrigine, pheneturide, phenacemide, valpromide, and valnoctamide; specific examples of antidiabetic agents include repaglinide, nateglinide, metformin, phenformin, rosiglitazone, pioglitazone, troglitazone, miglitol, acarbose, exanatide, vildagliptin, and sitagliptin; specific examples of blood glucose-lowering agent include tolbutamide, acetohexamide, tolazamide, glyburide, glimepiride, gliclazide, glipizide and chlorpropamide; specific examples of decongestants include pseudoephedrine, phenylephrine, and oxymetazoline; specific examples of antihistamines include mepyramine, antazoline, diphenhydramine, carbinoxamine, doxylamine, clemastine, dimenhydrinate, pheniramine, chlorpheniramine, dexchlorpheniramine, brompheniramine, tripolidine, cyclizine, chlorcyclizine, hydroxyzine, meclizine, promethazine, trimeprazine, cyproheptadine, azatadine, and ketotifen; specific examples of antitussives include dextromethorphan, noscapine, ethyl morphine, and codeine; specific examples of antineoplastics include chlorambucil, lomustine, tubulazole and echinomycin; specific examples of anti-inflammatory agents include betamethasone, prednisolone, aspirin, piroxicam, valdecoxib, carprofen, celecoxib, flurbiprofen and (+)-N-{4-[3-(4-fluorophenoxy)phenoxy]-2-cyclopenten-1-yl}-N-hyroxyurea; specific examples of beta-blockers include timolol and nadolol; specific examples of antitussives include dextromethorphan, noscapine, ethyl morphine, theobromine, and codeine; specific examples of anti-neoplastics include actinomycin, dactinomycin, doxorubicin, daunorubicin, epirurubicin, bleomycin, plicamycin, and mitomycin; specific examples of beta-blockers include alprenolol, carteolol, levobunolol, mepindolol, metipranolol, nadolol, oxprenolol, penbutolol, pindolol, propranolol, sotalol, timolol, acebutolol, atenolol, betaxolol, bisoprolol, esmolol, metoprolol, nebivolol, carvedilol, celiprolol, labetalol, and butaxemine; specific examples of antirheumatic agents include adalimumab, azathioprine, chloroquine, hydroxychloroquine, cyclosporine, D-penicillamine, etanercept, sodium aurothiomalate, auranofin, infliximab, leflunomide, methotrexate, minocycline, sulfasalazine; specific examples of anti-inflammatories include steroidal and nonsteroidal anti-inflammatory drugs such as hydrocortisone, prednisone, prednisolone, methylprednisolone, dexamethasone, betamethasone, triamcinolone, beclomethasone, aldosterone, acetaminophen, amoxiprin, benorilate, diflunisal, faislamine, diclofenac, aceclofenac, acemetacin, bromfenac, etodolac, indomethacin, nabumetone, sulindac, tolmetin, carprofen, ketorolac, mefenamic acid, phenylbutazone, aza anti-inflammatories, propazone, matamizole, oxyphenbutazone, sulfinprazone, piroxicam, lornoxicam, meloxicam, tenoxicam, celecoxib, etoricoxib, lumiricoxib, parecoxib, rofecoxib, valdecoxib, and numesulide; specific examples of antipsychotic agents include iloperidone, ziprasidone, olanzepine, thiothixene hydrochloride, fluspirilene, risperidone and penfluridole; a specific example of a cognitive enhancer includes ampakine; specific examples of anti-atherosclerotic, cardiovascular and/or cholesterol reducing agents include atorvastatin calcium, cerivastatin, fluvastatin, lovastatin, mevastatin, pitavastatin, pravastatin, rosuvastatin, and simvastatin; specific examples of antiobesity agents include dexadrine, dexfenfluramine, fenfluramine, phentermine, orlistat, acarbose, and rimonabant; specific examples of anti-impotence agents include sildenafil and sildenafil citrate; specific examples of anti-infective agents such as antibacterial, antiviral, antiprotozoal, antihelminthic and antifungal agents include carbenicillin indanyl sodium, bacampicillin hydrochloride, troleandomycin, doxycyline hydrate, ampicillin, penicillin G, azithromycin, oxytetracycline, minocycline, erythromycin, clarithromycin, spiramycin, acyclovir, nelfinavir, virazole, benzalkonium chloride, chlorhexidine, econazole, terconazole, fluconazole, voriconazole, griseofulvin, metronidazole, thiabendazole, oxfendazole, morantel, cotrimoxazole; specific examples of hypnotic agents include alfaxalone and etomidate; specific examples of anti-Parkinsonism agents include levodopa, bromocriptine, pramipexole, ropinirole, pergolide, and selegiline; anticholinergics such as trihexyphenidyl, benztropine mesylate, procyclidine, biperiden, andethopropazine; antihistamines such as diphenhydramine and dorphenadrine; and amantadine; specific examples of anti-Alzheimer's disease agents include donepezil rivastigmine, galantamine, tacrine; specific examples of antibiotics include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, telcoplanin, mupirocin, azithromycin, clarithromycin, ofloxacin, lomefloxacin, norfloxacin, nalidixic acid, sparfloxacin, pefloxacin, amifloxacin, enoxacin, fleroxacin, ternafloxacin, tosufloxacin, clinafloxacin, sulbactam, clavulanic acid, amphotericin B, fluconazole, itraconazole, ketoconazole, nystatin; specific examples of anti-depressants include isocarboxazid; phenelzine; tranylcypromine; specific examples of antiviral agents include azidovudine (AZT), didanosine (dideoxyinosine, ddI), d4T, zalcitabine (dideoxycytosine, ddC), nevirapine, lamivudine (epivir, 3TC), saquinavir (Invirase), ritonavir (Norvir), indinavir (Crixivan), delavirdine (Rescriptor); specific examples of glycogen phosphorylase inhibitors include [R—(R*S*)]-5-chloro-N-[2-hydroxy-3-{methoxymethylamino}-3-oxo-1-(phenylmethyl)propyl-1H-indole-2-carboxamide and 5-chloro-1H-indole-2-carboxylic acid [(1S)-benzyl-(2R)-hydroxy-3-((3R,4S)-dihydroxy-pyrrolidin-1-yl-)-3-o-xypropyl]amide; specific examples of cholesterol ester transfer protein inhibitors include [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, [2R,4S]4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R,4S]4-[(3,5-Bis-trifluoromethyl-benzyl)-methoxycarbonyl-amino]-2-eth-yl-6-trifluoromethyl-3,4-dihydro-2H-quinoline-1-carboxylic acid isopropyl ester; specific examples of CNS stimulants include caffeine and methylphenidate; specific examples of dopamine receptor agonists include cabergoline and pramipexole; specific examples of antiemetics include dolasetron, granisetron, ondansetron, tropisetron, palonosetron, domperidone, droperidol, dimenhydrinate, haloperidol, chlorpromazine, promethazine, prochlorperizine, metoclopramide, and alizapride; specific examples of gastrointestinal agents include loperamide and cisapride; specific examples of psychotherapeutic agents include chlorpromazine, thioridazine, prochlorperizine, haloperidol, alprazolam, amitriptyline, bupropion, buspirone, chlordiazepoxide, citalopram, clozapine, diazepam, fluoxetine, fluphenazine, fluvoxamine, hydroxyzine, lorezapam, loxapine, mirtazepine, molindone, nefazodone, nortriptyline, olanzepine, paroxetine, phenelzine, quetiapine, risperidone, sertraline, thiothixene, tranylcypromine, trazodone, venlafaxine, and ziprasidone; specific examples of opioid agonists include hydromorphone, fentanyl, methadone, morphine, oxycodone, and oxymorphone; specific examples of opioid antagonists include naltrexone; specific examples of anti-epileptic drugs include sodium valproate, nitrazepam, phenyloin; specific examples of histamine H₂ antagonists include famotidine, nizatidine, cimetidine, ranitidine; specific examples of anti-asthmatic agents include albuterol, montelukast sodium; specific examples of smooth muscle relaxants include nicorandil, iloperidone, and clonazepam; and specific examples of skeletal muscle relaxants include diazepam, lorazepam, baclofen, carisoprodol, chlorzoxazone, cyclobenzaprine, dantrolene, metaxalone, orphenadrine, pancuronium, tizanidine, dicyclomine, clonidine, and gabapentin. Each named drug should be understood to include the neutral form of the drug, as well as pharmaceutically acceptable salts, solvates, esters, and prodrugs thereof.

In one embodiment, the compositions of the present invention are orally disintegrating tablet (ODT) compositions comprising a therapeutically effective amount of a drug (e.g., temazepam), 0.5-3% of an ODT binder polymer, a sugar alcohol and/or saccharide, and a disintegrant.

In another embodiment, the ODT compositions of the present invention comprise a therapeutically effective amount of temazepam (i.e., 7-chloro-1,3-dihydro-3-hydroxy-1-methyl-5-phenyl-2H-1,4-benzodiazepin-2-one). Therapeutically effective amounts of temazepam range from 5-50 mg of temazepam/unit dose, for example 5, 7.5, 10, 12.5, 15, 17.5, 20, 22.5, 25, 27.5, 30, 32.5, 35, 37.5, 40, 42.5, 45, 47.5, or 50 mg temazepam/unit dose.

In one embodiment, the amount of temazepam per unit dose is 7.5, 15, 22.5, or 30 mg temazepam.

In other embodiments, the ODT compositions of the present invention comprise a therapeutically effective amount at least one drug. The term “therapeutically effective amount” refers to the amount of drug required to obtain one or more desired pharmacological effects. The therapeutically effective amount of a drug is conventionally determined by dosing trials, etc. using methods well know in the pharmaceutical arts.

The compositions of the present invention comprise particles of at least one drug of any suitable morphology (e.g., crystalline or amorphous), preferably morphologies which remain stable under processing and storage conditions. In one embodiment, the drug is present in crystalline form. The drug crystals can have an average particle size ranging from about 1 μm to about 200 μm, for example about 1-25 μm, about 1-50 μm, about 1-100 μm, about 1-150 μm, about 25-50 μm, about 25-150 μm, about 50-100 μm, about 50-150 μm, about 100-150 μm, or about 100-200 μm.

The ODT binder polymers suitable for use in the compositions of the present invention include pharmaceutically acceptable water-soluble, alcohol-soluble, or acetone/water soluble polymers. A non-limiting list of such polymers includes e.g., polyvinylpyrrolidone (povidone or PVP), starches such as corn starch, polyethylene glycols, polyethylene oxide, hydroxypropylmethylcellulose (HPMC), methylcellulose, hydroxypropylcellulose (HPC), etc., and mixtures thereof. In one embodiment, the ODT binder polymer is HPC, for example Klucel® LF or EF. As described herein, ODT binder polymers are pharmaceutically acceptable water-soluble, alcohol-soluble, or acetone/water soluble polymers added to the granulation fluid during the granulation of drug-containing particles with other excipients such as a filler, a diluent and/or a disintegrant, thereby reducing or eliminating sticking of particles to the sides if the granulation apparatus during granulation, reducing or eliminating the level of temazepam fines, reducing or eliminating scoring of the ODT tablets during extended tableting runs, and improving the reproducibility and yield of the process, without compromising the pharmacokinetic attributes of the composition.

The selection of and amount of ODT binder polymer used in the compositions of the present invention is critical. In the compositions of the present invention, the amount of ODT binder polymer ranges from 0.5-3% of the total weight of the binder-containing particle. In one embodiment, the amount of ODT binder polymer is less than about 2% by weight relative to the total weight of the binder-containing composition. In another embodiment, the amount of ODT binder polymer in the binder-containing composition is about 0.5-2%, 0.5-1.5%, 0.5-1%, less than about 1%, 1-3%, about 1-2.5%, about 1-2%, or about 1.5-2%, inclusive of all values, ranges, and subranges therebetween.

When the total amount of ODT binder polymer in the binder-containing composition is below 0.5%, it becomes difficult to control the uniformity (i.e., amount of drug in a unit dose) and overall production yield of the orally disintegrating tablet compositions because the ingredients of the orally disintegrating tablet composition stick to the walls of the manufacturing equipment (e.g., the walls of a fluidized bed coating apparatus). In addition, when the level of the ODT binder polymer in the binder-containing composition is below 0.5%, granulation results in an undesirable bimodal particle size distribution and the production of excessive amounts of “fines” (i.e., very small particles), which results in erratic flow properties, etc, and “scoring” or striation of the tablets by the “fines” after long tableting runs (e.g., greater than one hour). When the amount of ODT binder polymer in the binder-containing composition exceeds 3%, granulation results in larger and/or harder agglomerates which are unsuitable for oral dosage forms, e.g. because large agglomerates have an unpalatable “mouth feel” in orally disintegrating tablet (ODT) formulations and because larger agglomerates tend to reduce the release rate of the drug. However, adding an additional milling step to reduce the particle size of such agglomerates and obtain commercially acceptable production yields is hazardous for compositions which contain drugs, such as temazepam, which have a high explosion potential. Compositions according to the present invention with ODT binder polymer ranges of 0.5-3% have significantly improved processing properties and avoid the need to discard or mill large agglomerates.

An ODT dosage form differs from other oral dosage forms (e.g., conventional tablets, capsules, etc.) in that ODT dosage forms disintegrate at a significantly faster rate after administration. ODT compositions of the present invention contain pharmaceutically acceptable ingredients which swell, dissolve or otherwise facilitate the disintegration of the ODT composition in the oral cavity. Such ingredients can include pharmaceutical disintegrants, fillers/diluents, water-soluble binders, meltable solids (e.g., waxes), which can release the temazepam upon entering the stomach, etc.

The ODT compositions of the present invention disintegrate essentially completely in about 60 seconds or less, about 50 seconds or less, about 40 seconds or less, about 30 seconds or less, about 20 seconds or less, or about 10 seconds or less after administration. In one embodiment, the ODT compositions of the present invention release about 80% or more of the drug (e.g., temazepam) in 30 min. or less when tested for dissolution using USP official methodology (USP Apparatus 2; paddles @ 75 rpm in 900 mL of sodium acetate buffer (pH 4.0) with 0.05% polysorbate 80; detection: UV absorption at 310 nm). In another embodiment, the ODT compositions of the present invention release about 70% or more of the drug (e.g., temazepam) in 30 min. or less when tested for dissolution using USP official methodology (USP Apparatus 2; paddles @ 75 rpm in 900 mL of sodium acetate buffer (pH 4.0) with 0.05% polysorbate 80; detection: UV absorption at 310 nm).

In addition to the drug (e.g., temazepam) and ODT binder polymer, the ODT compositions of the present invention further comprise at least one disintegrant, and at least one sugar alcohol and/or saccharide to facilitate rapid disintegration of the ODT in the oral cavity.

Disintegrants suitable for use in the orally disintegrating tablet compositions of the present invention include any pharmaceutically acceptable disintegrants or “super-disintegrants”. A non-limiting list of suitable disintegrants includes crospovidone (crosslinked PVP), sodium starch glycolate, crosslinked sodium carboxymethylcellulose, and low-substituted hydroxypropylcellulose.

Sugar alcohols suitable for use in the orally disintegrating tablet compositions of the present invention include any pharmaceutically acceptable sugar alcohols. A non-limiting list of suitable sugar alcohols includes, e.g. mannitol, sorbitol, xylitol, maltitol, lactitol, erythritol, isomalt, hydrogenated starch hydrolysates, and maltol.

Saccharides suitable for use in the orally disintegrating tablet compositions of the present invention include any pharmaceutically acceptable saccharides. A non-limiting list of suitable saccharides includes, e.g. lactose, sucrose, and maltose.

The disintegrants and sugar alcohols and/or saccharides can be mixed (e.g., granulated) with the drug (e.g., temazepam) and ODT binder polymer, or the disintegrants and sugar alcohols and/or saccharides can be granulated together to form rapidly-dispersing microgranules, for example as described in U.S. patent application Ser. Nos. 10/827,106, 09/147,374, 10/356,641, 10/469,915 and 10/506,349, and EP 0914 818, each of which is herein incorporated by reference in its entirety for all purposes. In one embodiment, the rapidly-dispersing microgranules comprise at least one disintegrant in combination with at least one sugar alcohol. In another embodiment, the rapidly-dispersing microgranules comprise at least one disintegrant in combination with at least one saccharide. In yet another embodiment, the rapidly dispersing microgranules comprise at least one disintegrant in combination with at least one sugar alcohol and at least one saccharide. The amount of sugar alcohol and/or saccharide in the rapidly-dispersing microgranules ranges from about 90-99%, or about 95-99% of the total weight of the rapidly-dispersing microgranules. Alternatively expressed, the amount of a disintegrant in the rapidly-dispersing microgranules ranges from about 1-10%, or about 1-5%.

The average particle size of the sugar alcohol and/or saccharide as well as rapidly dispersing microgranules should be such that upon disintegration in the oral cavity, the composition provides a smooth mouthfeel (no aftertaste or gritty or chalky taste or texture). The average particle size of the sugar alcohol and/or saccharide in the rapidly-dispersing microgranules is 30 μm or less, for example about 1-30 μm, about 1-25 μm, about 1-20 μm, about 1-15 μm, about 1-10 μm, about 1-5 μm, about 5-30 μm, about 5-25 μm, about 5-20 μm, about 5-15 μm, about 5-10 μm, about 10-30 μm, about 10-25 μm, about 10-20 μm, about 10-15 μm, about 15-30 μm, about 15-25 μm, or about 15-20 μm, about 20-30 μm, or about 20-25 μm, inclusive of all values, ranges, and subranges therebetween. In one embodiment, the rapidly dispersing microgranules have an average particle size in the range of about 100-400 μm. In other embodiments, the average particle size of the rapidly dispersing microgranules is 300 μm or less. In a particular embodiment, the rapidly dispersing microgranules comprise mannitol and/or lactose and Crospovidone XL-10, wherein the ratio of mannitol and/or lactose to Crospovidone XL-10 in the rapidly dispersing microgranules ranges from about 99:1 to about 90:10. In another embodiment, the rapidly dispersing granules are prepared by wet granulating D-mannitol having an average particle size of about 15 μm with Crospovidone XL-10 at a ratio of about 95/5 in a high shear granulator using purified water as the granulating fluid, and drying the granules by spreading the granulated mixture on trays in a heated convection oven as described in U.S. Patent Publication No. 2005/0232988.

In yet another embodiment of the present invention, the ODT composition further comprises a mixture of at least one disintegrant and at least one sugar alcohol and/or saccharide, optionally granulated with an aqueous solution of an ODT binder polymer in the range of about 0.5-3% of the total weight of the binder-containing composition.

The pharmaceutical compositions of the present invention can further comprise additional pharmaceutically acceptable ingredients or excipients. Examples of suitable excipients for use in the compositions or dosage forms of the present invention include fillers, diluents, glidants, disintegrants, binders, lubricants etc. Other pharmaceutically acceptable excipients include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants include e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc. Specific examples of binders include e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Specific examples of glidants and lubricants include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients include e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Non-limiting examples of flavoring agents include e.g. cherry, orange, or other acceptable fruit flavors, or mixtures of cherry, orange, and other acceptable fruit flavors, at up to about 3% based on the tablet weight. In addition, the compositions of the present invention is can also include one or more sweeteners such as aspartame, sucralose, or other pharmaceutically acceptable sweeteners, or mixtures of such sweeteners, at up to about 2% by weight, based on the tablet weight. Furthermore, the compositions of the present invention can include one or more FD&C colorants at up to 0.5% by weight, based on the tablet weight.

Antioxidants include e.g. ascorbic acid, ascorbyl palmitate, butylated hydroxyanisole, butylated hydroxytoluene, hypophosphorous acid, monothioglycerol, potassium metabisulfite, propyl gal late, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc.

In a particular embodiment, ODT compositions of the present invention which contain rapidly dispersing microgranules can include an additional disintegrant, in addition to the disintegrant in the rapidly dispersing granules. The additional disintegrant can be the same or different from the disintegrant used in the rapidly dispersing granules. The additional disintegrant may be present in the ODT compositions of the present invention at up to about 10% based on the total tablet weight.

The optional pharmaceutically acceptable excipients can be incorporated into temazepam-containing particles, temazepam-containing microgranules, temazepam-layered beads, rapidly-dispersing microgranules, or can be added in addition to the temazepam-containing particles, temazepam-containing microgranules, temazepam-layered beads, or rapidly-dispersing microgranules. In some cases, the optional pharmaceutically acceptable excipients can include ingredients also present in the temazepam-containing particles, temazepain-containing microgranules, temazepam-layered beads, or rapidly-dispersing microgranules. For example, temazepam-containing microgranules and rapidly-dispersing microgranules (which include a disintegrant) can be blended with a disintegrant which is either the same or different from the disintegrant present in the rapidly-dispersing microgranules.

In one embodiment, the compositions of the present invention comprise, in addition to the drug (e.g., temazepam), binder, sugar alcohol and/or saccharide, and disintegrant, microcrystalline cellulose (e.g., Avicel PH101, Avicel PH102, Ceolus KG-802 or KG-1000, Prosolv SMCC 50, SMCC 90, or mixtures thereof).

The amount of optional pharmaceutically acceptable ingredients (e.g. fillers or diluents and disintegrants) in the drug-containing microgranules can range from about 5%-80%, including about 5%-70%, about 5%-60%, about 5%-50%, about 5%-40%, about 5%-30%, about 5%-20%, about 5%-15%, about 5%-10%, about 10%-70%, about 10%-60%, about 10%-50%, about 10%-40%, about 10%-30%, about 10%-20%, about 10%-15%, about 20%-70%, about 20%-60%, about 20%-50%, about 20%-40%, about 20%-30%, about 20%-25%, about 30%-70%, about 30%-60%, about 30%-50%, about 30%-40%, about 30%-35%, about 40%-70%, about 40%-60%, about 40%-50%, about 40%-45%, about 50%-70%, about 50%-60%, about 50%-55%, about 60%-70%, or about 60%-65%.

The compositions of the present invention can comprise at least one drug (e.g., temazepam), one or more ODT binder polymers, one or more disintegrants, and one or more sugar alcohols; at least one drug (e.g., temazepam), one or more ODT binder polymers, one or more disintegrants, and one or more saccharides; or at least one drug (e.g., temazepam), one or more ODT binder polymers, one or more disintegrants, one or more sugar alcohols, and one or more saccharides. In one embodiment, the compositions of the present invention comprise at least one drug (e.g., temazepam), an ODT binder polymer, disintegrant, a sugar alcohol. In another embodiment, the compositions of the present invention comprise at least one drug (e.g., temazepam), hydroxypropylcellulose, crospovidone, and mannitol.

For example, the compositions of the present invention can comprise a mixture of drug particles and rapidly-dispersing microgranules, compressed into the form of an orally disintegrating tablet. The drug particles can be e.g. drug crystals, drug microgranules prepared by (wet or dry) granulating the drug in the presence of one or more ODT binder polymers, and optionally additional pharmaceutically acceptable excipients. Or, the drug particles can be drug-layered beads prepared by dissolving or suspending the drug in a solution of one or more drug-layering binders, coating the resulting solution or suspension onto inert core particles (e.g., sugar spheres such as Celphere® CP-203 of 50-100 mesh or 150-300 μm, cellulose spheres, silicon dioxide spheres, etc.), and removing the solvent.

Suitable drug layering binders include any of the ODT binder polymers disclosed herein, for example starches, modified celluloses (e.g., hydroxypropylcellulose, carboxymethylcellulose sodium), alginic acid, polyvinyl pyrrolidone (povidone), and mixtures thereof. The amount of drug in the drug layer, and the thickness of the drug layer can be modified to provide a therapeutically effective dose of the drug. The drug-containing layer comprises about 90% to about 99% drug and about 10% to about 1% of the binder, and the drug-containing layer is about 10% to about 30% of the total weight of the drug-layered beads.

The drug (e.g., temazepam) particles (e.g., crystals, microgranules or drug-layered beads) of ODT compositions of the present invention have an average particle size of 1-400 μm. Drug crystals with an average particle size range of about 1-200 μm, in some embodiments about 50-150 μm, are coated with a taste-masking layer by either fluid bed coating or coacervation as described herein. In other embodiments, drug crystals with a mean particle size of about 5-50 μm are taste-masked by solvent coacervation as described herein.

As described herein, the drug particles can contain additional excipients. When the drug particles contain additional excipients (e.g. fillers or diluents and disintegrants), the amount of such additional excipients in the drug particles can range from about 5%-80%, including about 5%-70%, about 5%-60%, about 5%-50%, about 5%-40%, about 5%-30%, about 5%-20%, about 5%-15%, about 5%-10%, about 10%-70%, about 10%-60%, about 10%-50%, about 10%-40%, about 10%-30%, about 10%-20%, about 10%-15%, about 20%-70%, about 20%-60%, about 20%-50%, about 20%-40%, about 20%-30%, about 20%-25%, about 30%-70%, about 30%-60%, about 30%-50%, about 30%-40%, about 30%-35%, about 40%-70%, about 40%-60%, about 40%-50%, about 40%-45%, about 50%-70%, about 50%-60%, about 50%-55%, about 60%-70%, or about 60%-65%.

In one embodiment, the ODT compositions of the present invention comprise drug (e.g., temazepam) particles in combination with an ODT binder polymer and rapidly dispersing granules. The amount of rapidly dispersing granules in the ODT compositions of the present invention can vary depending upon the desired disintegration time, organoleptic properties and the desired dose of the drug, and can range from about 50% to about 90%, including about 50%-80, about 50%-70, about 50%-60, about 60%-80, about 60%-70, or about 70%-80%, inclusive of all values, ranges, and subranges therebetween.

Likewise, the ODT compositions of the present invention should contain a sufficient quantity of drug particles to provide a therapeutically effective dose of the drug. The amount of drug in the drug particles can range from about 5% to about 50%, including about 5-40%, about 5-30%, about 5-20%, about 5-10%, about 10-40%, about 10-30%, about 10-20%, about 20-40%, and about 20-30%, inclusive of all values, ranges, and subranges therebetween.

The compositions of the present invention can be prepared by granulating a mixture of the drug, one or more ODT binder polymers, and rapidly-dispersing microgranules (as described above), or by granulating a mixture of the drug, one or more ODT binder polymers, one or more disintegrants, and one or more sugar alcohols and/or saccharides. The granulated mixtures are then compressed When the compositions of the present invention comprise drug particles (e.g. temazepam microgranules or temazepam-layered beads), the drug particles can be coated with a taste-masking layer, such as those described in U.S. Patent Publication Nos. 2006/0105038, 2006/0078614, and 2006/0105039, herein incorporated by reference in their entirety for all purposes. The taste masking layer, when present, comprises one or more water-insoluble polymers. Non-limiting examples of suitable water-insoluble polymers include, e.g., ethyl cellulose (e.g., ETHOCEL Standard Premium from Dow), polyvinyl acetate (PVA), cellulose acetate (CA), cellulose acetate butyrate (CAB), and methacrylate copolymers available under the tradename “EUDRAGIT” (such as Eudragit RL, Eudragit RS, Eudragit NE30D, etc.). In a particular embodiment, the water-insoluble polymer is ethyl cellulose having a viscosity in the range of 7-100 cps (measured as a 5% solution in 80/20 toluene/ethanol at 25° C. in an Ubbelohde viscometer).

In other embodiments, the taste masking layer comprises a mixture of one or more water-insoluble polymers in combination with a pore former which is insoluble in water and saliva but is readily soluble under acidic conditions (e.g., in the stomach). Suitable pore formers include, for example, gastrosoluble oxides, hydroxides and salts of organic and inorganic acids, as well as gastrosoluble pore forming polymers. A non-limiting list of suitable pore-formers include calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, and the like, polymers of the EUDRAGIT® E series (e.g., EUDRAGIT® E100 or EUDRAGIT® EPO) or a poly(vinylacetal diethylaminoacetate) e.g., AEA® available from Sankyo Company Limited, Tokyo (Japan), and mixtures thereof. The ratio of water-insoluble polymer to pore former typically varies from about 95/5 to about 50/50, or in some embodiments from about 85/15 to 65/35.

Because coating the drug-containing particles (e.g., temazepam crystallites, microgranules or drug-layered beads) with a water-insoluble polymer may reduce the release-rate of the drug, the taste-masking coating can also include a pore-former. Pore-formers include gastrosoluble pore-formers which are insoluble in water and saliva, but are readily soluble under acidic conditions, such as those found in the stomach, and water-soluble pore-formers. Non-limiting examples of suitable gastrosoluble pore-formers include, e.g. calcium carbonate, magnesium citrate, and magnesium hydroxide. Non-limiting examples of suitable water-soluble pore-formers include, e.g. sodium chloride, sucrose, and povidone. The ratio of water-insoluble polymer to pore-former in the taste-masking layer can range from about 95/5 to about 50/50. The taste-masking layer can range from about 5% to about 30% of the total weight of the taste-masked temazepam-containing particle, or about 5%-25%, about 5%-20%, about 5%-15%, about 5%-10%, about 10%-30%, about 10%-25%, about 10%-20%, about 10%-15%, about 15%-30%, about 50%-25%, about 15%-20%, about 20%-30%, about 20%-25%, or about 25%-30% of the total weight of the of the taste-masked temazepam-containing particle.

In one embodiment, the ODT compositions of the present invention are prepared by (a) forming drug particles (e.g., temazepam-containing granules, microencapsulated temazepam crystals, temazepam-layered beads, etc.); (b) blending the drug particles with a disintegrant, a sugar alcohol and/or saccharide, and 0.5-3% of an ODT binder (based on the total weight of the ODT composition); and (c) compressing the blend into an ODT.

In one embodiment, step (a) above, forming drug particles, is granulating the drug with other excipients (e.g., a sugar alcohol such as mannitol). The granulation step can be carried out under wet or dry conditions, depending on the excipients used, in conventional granulation equipment, such as fluid bed granulators available from Glatt and Fluid Air. In a specific embodiment, the drug particles are prepared by wet granulating the drug and mannitol. In another embodiment, step (a) is microencapsulating drug crystals by fluidized bed coating or coacervation, for example with a taste-masking or seal coating composition. In yet another embodiment, step (a) is forming drug-layered beads by coating an inert core, such as sugar spheres, with a solution or dispersion of the drug and a drug-layering binder such as PVP.

The disintegrant and sugar alcohol and/or saccharide in step (b) above, can be separately added to the drug (e.g., teinazepam) particles, then mixed, or alternatively can be combined to form rapidly disintegrating granules, which are then mixed with the drug particles. In one embodiment, step (b) is blending the drug particles with rapidly disintegrating granules prepared by wet granulation (e.g., using water as the granulating fluid) or dry granulation at least one disintegrant with at least one sugar alcohol and/or saccharide in a fluid bed granulator. In a specific embodiment, the rapidly disintegrating granules comprise crospovidone and mannitol. In other embodiments, the drug (e.g., temazepam) particles, disintegrant, and sugar alcohol and/or saccharide are granulated together, optionally with additional excipients such as compression aids (e.g., microcrystalline cellulose) or lubricants (e.g., magnesium stearate). In one embodiment, crystalline temazepam, a sugar alcohol, a disintegrant, and a compression aid are granulated with a solution of a water-soluble ODT binder. In another particular embodiment, crystalline temazepam, mannitol, crospovidone and microcrystalline cellulose are granulated with an aqueous solution of hydroxypropyl cellulose.

The compressing step (c) above can be carried out using conventional methods, for example with a rotary tablet press or an externally lubricated tablet press.

Alternative processes which omit the ODT binder (during granulation of the drug, disintegrant, sugar alcohol and/or saccharide, and optional additional excipients) do not provide commercially acceptable ODTs, in acceptable yield. For example, in a process in which D-mannitol having a mean particle size of not more than 35 μm, crospovidone, and temazepam was granulated with rapidly dispersing microgranules comprising mannitol and crospovidone and other excipients (peppermint flavor, a sweetener and additional disintegrant), significant sticking of the granulated material occurred on the sides of the fluid bed granulator, resulting in low product yield, and a bimodal distribution of microgranules containing a significant fraction of fines. The sticking problem also resulted in the production of ODT product with a mottled appearance (due to the inconsistent distribution of ingredients in the tablet), inconsistent levels of drug (e.g., temazepam) and scoring (striations) on the tablets during compression caused by the high level of fines.

Surprisingly, the addition of the ODT binder to the compositions of the present invention significantly reduces the fines responsible for the observed scoring, and decreases the quantity of material sticking to the sides of the fluid bed granulator, thereby increasing the product yields and the overall compositional uniformity of the ODT, particularly in long production runs. In addition, the selection and concentration of the ODT binder is critical to avoiding the creation of larger and/or harder agglomerates which would require milling to achieve higher useable yield (increased productivity), and to maintaining dissolution rates under in vitro/in vivo conditions which provide bioequivalence to conventional oral dosage forms (e.g., comparing the dissolution rates of temazepam-containing ODT compositions of the present invention with the reference listed drug product, Restoril®, temazepam immediate release capsules).

As described herein, it was found that the exposed drug particles, particularly the fines, were responsible for the observed scoring and striation of the ODT tablets observed during long tableting runs (typical of commercial production conditions). By using a small amount of ODT binder polymer in the granulating fluid, it was surprisingly found that the quantity of material sticking to the sides of the fluid bed granulator could be substantially reduced, and the scoring observed on the tablets during compression could also be substantially reduced.

In addition, in view of sensitivity of some drugs to processing conditions (e.g., the high explosion potential of temazepam), the selection and concentration of the ODT binder polymer was found to be critical in order to avoid creating larger and/or harder agglomerates which would require milling to achieve higher useable yield and which would reduce the bioavailability of the drug (due to the slower dissolution of the larger agglomerated).

Thus, in a specific embodiment, the ODT compositions of the present invention are prepared by granulating a composition comprising about 10-15% by weight temazepam, a sugar alcohol (e.g., mannitol), and a disintegrant (e.g., crospovidone) in the presence of a solution of a hydrophilic ODT binder polymer at a relatively low concentration (e.g., less than about 2% by weight relative to the weight of the ODT), and compressing this composition into orally disintegrating tablets. The resulting ODT is robust enough to be packaged into blisters or bottles for storage, transportation and commercialization, has good organoleptic properties upon administration, rapid in vitro disintegration, and rapid dissolution/drug release.

In another embodiment, the ODT compositions of the present invention are prepared by: (a) preparing drug-containing microgranules by granulating crystalline drug having an average particle size of about 1-50 μm and one or more diluents/fillers such as lactose, mannitol, microcrystalline cellulose and mixtures thereof, with a polymeric binder in a high-shear granulator or a fluid-bed coater; (b) granulating one or more sugar alcohols, saccharides, or mixtures thereof, each having an average particle diameter of not more than about 30 μm, with a disintegrant such as crospovidone, using water or an alcohol-water mixture in a conventional granulator, and drying the granulate in fluid-bed equipment or a conventional oven to produce ‘rapidly-dispersing microgranules’ with an average particle size of not more than about 400 μm; (c) blending the drug microgranules of step (a) with one or more flavoring agents, a sweetener, microcrystalline cellulose, additional disintegrant, and the rapidly-dispersing microgranules of step (b); and (d) compressing the blend of step (c) into tablets using e.g. a conventional rotary tablet press equipped with an external lubrication system to pre-lubricate the dies and punches.

In another embodiment, the ODT compositions of the present invention are prepared by: (a) preparing a drug-containing core particle (e.g., temazepam crystallites, drug-layered beads, or temazepam-containing microgranules) by granulating the drug and one or more diluents/fillers such as lactose, mannitol, microcrystalline cellulose and mixtures thereof with an ODT binder polymer in a high-shear granulator or a fluid-bed coater, or drug-layering on an inert particle (60-100 mesh sugar sphere or cellulose sphere, e.g., Celphere® CP-203) from a solution/suspension comprising a polymeric binder and the drug in a fluid-bed coater and optionally applying with a seal-coat (e.g., Opadry® Clear); (b) taste-masking core particles by microencapsulation, e.g. by solvent coacervation or fluid-bed coating with a water-insoluble polymer such as ethylcellulose, or with a mixture of a water-insoluble functional polymer and a water-soluble/gastrosoluble pore-former (e.g., ethylcellulose and sodium chloride or calcium carbonate at a ratio ranging from about 50/50 to 95/5) to produce pleasant-tasting microparticles with a desired particle size distribution (e.g., an average particle size of not more than about 400 μm, or an average particle size of not more than about 300 μm); (c) granulating one or more sugar alcohols, saccharides or mixtures thereof, each of which has an average particle diameter of not more than about 30 μm, with a disintegrant such as crospovidone, as disclosed herein; (d) blending the taste-masked microparticles of step (b) with one or more flavoring agents, a sweetener, microcrystalline cellulose, additional disintegrant, and rapidly-dispersing microgranules of step (c); and compressing the blend of step (d) into tablets using e.g. a conventional rotary tablet press equipped with an external lubrication system to pre-lubricate the dies and punches.

### In Vitro Disintegration Time/Dissolution Testing:

Disintegration times were measured using the USP <701> Disintegration Test procedures. The taste-masking property of the taste-masked microparticles and the orally disintegrating tablets can be evaluated by placing the taste-masked microparticles or the orally disintegrating tablet on the tongue of a test subject, moving the taste-masked microparticles (after disintegration of the orally disintegrating tablet) around in the subject's oral cavity, and noting the perceived drug taste or aftertaste, if experienced. In addition, the rapid-release property in the stomach of the taste-masked microparticles and the orally disintegrating tablets can be evaluated by determining the percentage of drug-release when tested by the USP official methodology (USP Apparatus 2; paddles @ 75 rpm in 900 mL of sodium acetate buffer (pH 4.0) with 0.05% polysorbate 80; detection: UV absorption at 310 nm) (a release of not less than about 75% of the dose in about 30 minutes is considered acceptable).

The rate of disintegration of ODT compositions in the oral cavity of a patient can be on the order of about 60 seconds or less, about 50 seconds or less, about 40 seconds or less, about 30 seconds or less, about 20 seconds or less, or about 10 seconds or less.

Alternatively, the rate of disintegration can be measured using various in vitro test methods, for example the USP <701> Disintegration Test while the ODT is tested for dissolution using the United States Pharmacopoeia official methodology (USP Apparatus 2; paddles @ 75 rpm in 900 mL of sodium acetate buffer (pH 4.0) with 0.05% polysorbate 80; detection: UV absorption at 310 nm). When using the USP <701> Disintegration Test, the rate of disintegration of ODT compositions is faster than that of conventional, non-ODT compositions, for example 60 seconds or less, 30 seconds or less, 20 seconds or less, or 10 seconds or less. When using the United States Pharmacopoeia official methodology, the rate of release of the drug (e.g., temazepam) is comparable, may be slightly faster or slower than that of conventional, non-ODT compositions, for example about 70%, about 75%, about 80%, about 85%, about 90%, about 95%, or about 100% temazepam released in 30 min.

As discussed above, because ODT compositions disintegrate in the mouth of the patient, ODT compositions must be palatable, that is, have acceptable organoleptic attributes such as taste, after-taste, “mouthfeel”, in vitro disintegration time (DT), oral or in vivo DT, and Flavor-Sweetener Balance (FSB). The taste and after-taste attributes are defined using a bitterness scale of 1 to 10, i.e., 1 (extremely bitter; taste-masking required), 5 (acceptable), and 10 (pleasant tasting). The mouthfeel scale ranges from 1 (very gritty, unacceptable), 5 (non-gritty), and 10 (creamy, smooth). The FSB scale ranges from 4 (low, need to increase), 7 (optimum), and 10 (over-powered, need to reduce).

The ODT compositions of the present invention have taste and aftertaste attributes at least about 5, at least about 6, at least about 7, at least about 8, at least about 9, or about 10. The ODT compositions of the present invention have mouthfeel attributes of at least about 5, at least about 6, at least about 7, at least about 8, at least about 9, or about 10. The ODT compositions of the present invention have FSB attributes of about 5 to about 9, or about 6 to about 8, or about 7.

The ODT compositions of the present invention provide acceptable taste-masking from the time the ODT composition is placed in the mouth until swallowed. The ODT compositions of the present invention disintegrate in about 60 seconds or less when evaluated using the USP <701> Disintegration Test, and typically disintegrate on contact with saliva in the buccal cavity in about 60 seconds, forming a smooth, easy-to swallow suspension of taste-masked microparticles with an acceptable aftertaste. The taste-masked microparticles provide a substantially complete release of the temazepam dose upon entering the stomach (e.g., not less than about 60%, more particularly not less than 70% of the total dose released in about 30 minutes when tested for dissolution by the USP official dissolution methodology (USP Apparatus 2; paddles @ 75 rpm in 900 mL of sodium acetate buffer (pH 4.0) with 0.05% polysorbate 80; detection: UV absorption at 310 nm).

Compositions of the present invention (e.g. ODT compositions) comprising drug-containing (e.g., temazepam) microgranules in accordance with one embodiment exhibit the following properties:
acceptable hardness and friability suitable for packaging in bottles and blister packaging, storage, transportation and commercial distribution;
disintegration on contact with saliva in the oral cavity in about 60 seconds forming a smooth, easy-to-swallow suspension with a pleasant taste (no grittiness or aftertaste), meeting the specification of not more than 60 seconds, more particularly not more than 30 seconds in the <USP 701> Disintegration Test; and
substantially complete release of the temazepam upon entry into the stomach, as shown by the release of not less than about 75% of the dose in about 30 minutes in the official dissolution media (USP Apparatus 2; paddles @ 75 rpm in 900 mL of sodium acetate buffer (pH 4.0) with 0.05% polysorbate 80; detection: UV absorption at 310 nm).

In one embodiment, the compositions of the present invention contain a therapeutically effective amount of temazepam, can be administered according to any suitable dosage schedule which can be readily determined by a physician, and are suitable for the treatment of insomnia. In one embodiment, the present invention relates to a method of treating a patient with a sleeping disorder comprising the orally disintegrating tablet compositions described herein. In another embodiment, the present the present invention relates to a method of treating a patient suffering from dysphagia and a sleeping disorder comprising the orally disintegrating tablet compositions described herein.

The following non-limiting examples illustrate the ODT compositions of the present invention, and methods for preparing such compositions.

### EXAMPLES

### Example 1

### Example 1A

### Rapidly Dispersing Microgranules

Rapidly dispersing microgranules were prepared by mixing D-mannitol having an average particle size of about 15 μm and Crospovidone XL-10 at a ratio of about 95/5 in a high shear granulator using purified water as the granulating fluid. The resulting rapidly dispersing microgranules were dried by spreading the granulated mixture on trays in a heated convection oven. The average particle size of the dried rapidly dispersing microgranules was less than about 400 μm.

### Example 1B

### 30 mg Temazepam ODT

Sucralose, cherry or peppermint flavor, Crospovidone XL-10 and microcrystalline cellulose were pre-blended, then blended with crystalline temazepam and compressed into 30 mg tablets using a Hata tablet press and 11 mm, round, standard, concave, tooling, a vacuum transfer system, tablet de-duster, a metal detector, and a Matsui ExLub system. The ExLub system sprays lubricant (e.g., magnesium stearate) at a pre-selected rate into the die cavities and on punch surfaces and then vacuums off the excess prior to compression. The punch and die surfaces were externally lubricated with magnesium stearate, so the lubricant was therefore present in only trace amounts on the tablets. The tablet press settings were adjusted to provide tablets with a friability of less than 1% and a hardness of about 30 N by varying the compression forces from about 8 kN to about 16 kN. Relative standard deviations (RSD) of the tablet weights (target weight: 500 mg) were very low, ranging from 0.24% to 1.22%. Tablet hardness ranged from about 34N to about 129 N depending upon the compression force, and the friability ranged from 0.6% to 1.5% depending upon the tablet hardness. Tablet disintegration times were less than 30 seconds and the dissolution values were all >90% in 30 minutes using the USP official dissolution methodology (USP Apparatus 2; paddles @ 75 rpm in 900 mL of sodium acetate buffer (pH 4.0) with 0.05% polysorbate 80; detection: UV absorption at 310 nm). In spite of the tight tablet weight control, the temazepam assays were low, and highly variable content uniformity values were observed. The low temazepam assay values appear to be caused by the vacuum transfer system of the Hata press, which vacuumed off fine drug particles during pressing. Because of the low temazepam assay and the highly variable content uniformity values, this process was not considered to be commercially feasible.

### Example 2

### Example 2A

### Temazepam Microgranules

Temazepam microgranules were prepared by charging a Glatt GPCG 5 fluid bed granulator with temazepam, mannitol, and crospovidone, and granulating the mixture using purified water as a granulating fluid (batch size of 6 kg). Batches were made with about 6.3% w/w, 15.0% w/w and 30.0% w/w drug concentrations to evaluate the effect of increasing the concentration of temazepam on the quality of the resulting granules. Mannitol sticking to the sides of the fluid bed processor was observed during the manufacturing of these batches, resulting in a bi-modal particle size distribution and a significant fraction of fines, which in turn caused erratic flow properties.

### Example 2B

### Temazepam ODT (7.5, 15, 22.5 and 30 mg Temazepam Dosages)

Various ODT compositions were evaluated to determine the “robustness” of the formulations (e.g., the effect of the amounts of filler, disintegrant, sweetener, flavor, and rapidly dispersing microgranules, lubricant spray conditions, and tableting parameters such as compression force, fill depth, tablet weight, turret speed, etc. on ODT properties such as hardness, friability (target: <0.6%)). Temazepam compression batches were prepared by first pre-blending sucralose, cherry or peppermint flavor, crospovidone XL-10, and microcrystalline cellulose, then blending this mixture with the rapidly dispersing microgranules prepared as described in Example 1A, and the temazepam microgranules prepared as described above in Example 2A (having a drug content of either about 6.3% or about 30% by weight; see Table 1 for details), to provide temazepam compositions at a batch size of 0.5, 2, or 5 kg having a theoretical temazepam blend assay of 6.0% w/w in Table 2.

TABLE 1
Compositions of Temazepam Granules

| | Temazepam Granules | Temazepam Granules |
|---|---|---|
| Ingredient | (1136-JMC-029) | (1136-JMC-034) |
| Temazepam | 30.00% | 6.375% |
| Mannitol 25 | 65.00% | 88.625% |
| Crospovidone XL-10 | 5.00% | 5.00% |
| Granulating Fluid | 85.3% | 50.0% |
| (Water) | | |

TABLE 2
Compositions of Temazepam ODT Formulations

| Temazepam ODT | 1136-JMC-043 | | 1136-JMC-041 | |
|---|---|---|---|---|
| Temazepam Granules | 20.00% | | 94.12% | |
| Rapidly Dispersing Granules | 75.62% | | 1.50% | |
| Crospovidone XL-10 | 3.50% | | 3.50% | |
| Sucralose | 0.43% | | 0.43% | |
| NOR-CAP Peppermint | 0.35% | | 0.35% | |
| FD&C Blue# 1 | 0.10% | | 0.10% | |
| Total | 100.0 | | 100.0 | |
| % Particles <106 μm | 32.1% | | 39.0% | |
| Bulk Density | 0.60 | g/mL | 0.57 | g/mL |
| Tap Density (1120 taps) | 0.76 | g/mL | 0.60 | g/mL |
| Assay: Mean % (;) | 6.03% (5.96; 6.09) | | 6.28 (6.16; 6.39) | |
| Weight of 30 mg Dose | 500.0 | mg | 500.0 | mg |
| Weight of 7.5 mg Dose | 125.0 | mg | 125.0 | mg |

A compression batch (1136-JMC-041) prepared as described above was compressed on a Hata tablet press equipped with an ExLub external lubricating system into 7.5 mg and 30 mg tablets at a turret speed of 15 rpm (see Table 2 for details). 7.5-mg, 15-mg, 22.5, and 30-mg strength temazepam ODT tablets were also compressed using compression blend (1136-JMC-043) which were dose proportional to the 30 mg ODT tablets (see Table 3 for details). No flow-related problems or compression problems were observed during these short compression runs. The tablet weight variation, measured in terms of relative standard deviation (RSD) varied from 0.21% to 2.35%, and acceptable hardness and friability values were obtained. The disintegration times ranged from a low of 13 seconds to a high of 41 seconds. The relative standard deviations for the content uniformity varied from about 1.5% to 4.5%. The observed properties appeared to indicate that the processes to make the intermediate as well as the final compositions are sufficiently robust to produce ODTs at least at this manufacturing scale.

TABLE 3
Tableting and Analytical Data on Temazepam ODT Tablets

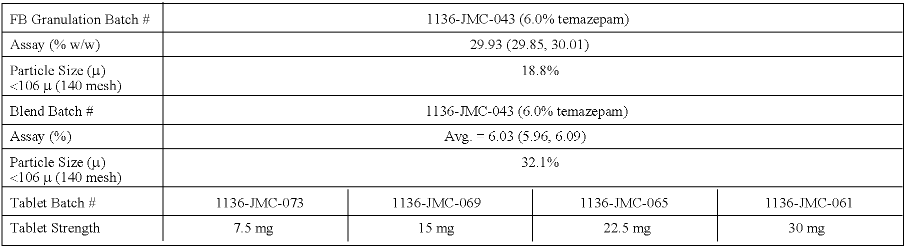

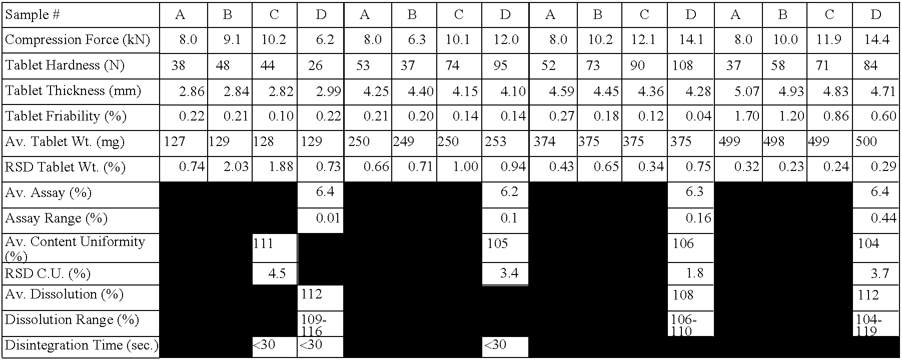

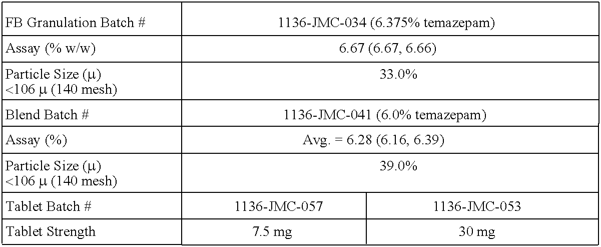

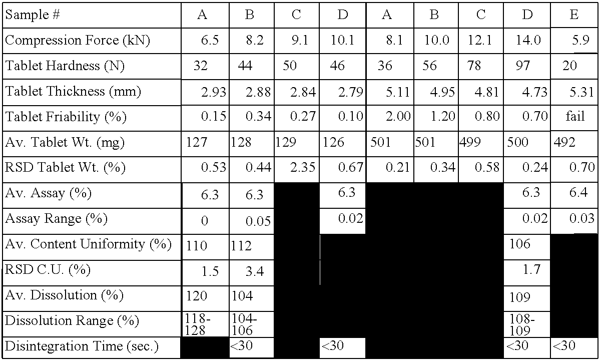

Note:
Shaded areas were not tested

### Example 2C

### Commercial Scale Processing

The temazepam granulation formulation developed using a Glatt gPCG 5 as described in Example 2B was scaled-up by preparing temazepam granules in a Fluid Air FA 0300 fluid bed granulator equipped with a 32″ top spray product bowl (batch size: 160 kg), 100 mesh bottom (product support) screen, three nozzles with a nozzle tip diameter of 0.085″, two pump heads/nozzles peristaltic pump settings of 6.4 mm, and dedicated 10 μm pore size filter bag. The Fluid Air product bowl was charged with 10.1 kg of temazepam, 140.0 kg of mannitol, and 8 kg of Crospovidone. These ingredients were granulated while being sprayed with an aqueous solution sucralose and FD & C Blue #1 (120 kg total: 0.56 kg Sucralose, 0.144 kg FD & C Blue #1, balance water). The following process parameters were used: inlet air temperature: 60-90° C., air volume: 1300 cfm (cubic feet per minute), spray rate set at 25%, product temperature at <50° C. Significant sticking of fine powder on the inner surface of the fluid-bed processor was observed.

The Fluid Air processed temazepam microgranules, rapidly dispersing microgranules prepared as described in Example 1A, and other excipients (sucralose, cherry or peppermint flavor, crospovidone XL-10 and microcrystalline cellulose) were blended in a 10 cu-ft V-blender (batch size: 150 kg), and compressed on a Hata tablet press equipped with an ExLub external lubrication system. The Hata tablet press was equipped with 11 mm round, flat face, radius edge “trade dress” tooling with an “R” monograph on one side. The turret speed was set at 25 rpm, and the magnesium stearate spray rate was set at approximately 1 to 2 volts (medium spray rate). Severe picking (i.e., defects due to contamination of the die by fines) was noted in the island of the “R” almost immediately. After about one hour of operation, scoring along the sides of the tablets was also observed. Some mottling was also observed in the tablet appearance, indicating uneven mixing of the components of the mixture. These problems were not observed during short compression runs of pilot scale batches.

In order to eliminate the problems identified during scale-up, several modifications to the processing conditions and ODT formulation were made, including addition of extra disintegrant, varying the turret speed, changing tooling configurations, varying the external lubricant spray rate, adding an internal lubricant to the ODT formulation. However, none of these process changes resolved the picking, scoring, and mottling problems. A large scale placebo batch (i.e., mannitol and crospovidone, but no temazepam) was also prepared in the FA 300 fluid bed granulator, and was blended with the remaining excipients (sucralose, cherry or peppermint flavor, crospovidone XL-10 and microcrystalline cellulose) and compressed on a Hata tablet press using the trade dress radius edge tooling, but no scoring was observed. Accordingly, the scoring observed during compression was attributed to the presence of exposed drug particles, particularly the drug particle fines produced during processing.

### Example 3

### Example 3A

### Temazepam Granules Containing an ODT Binder

A series of 6 small scale batches (see Table 4 for compositions of Temazepam Granules; batch size: 6 kg) were granulated in a Glatt 5 fluid bed granulator in the presence of small amounts of hydroxypropyl cellulose NF, following the same operating parameters used above in Example 2.

TABLE 4
Compositions of Reformulated Temazepam Granules

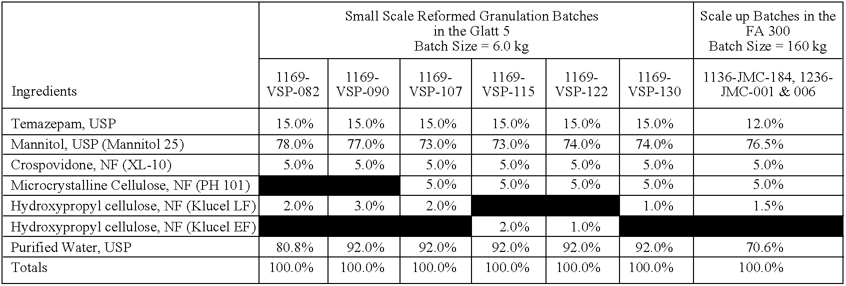

### Example 3B

### Scale-Up of Temazepam Granules

The composition of the temazepam granules is shown in Table 5, and the composition of the compression blend is shown in Table 5.

TABLE 5
Compositions of Temazepam Granulation and ODT
Compression Blend

| | | Compression |
|---|---|---|
| Ingredient (kg/Batch) | Microgranules | Blend |
| Temazepam Microgranules | | 70.0* |
| Temazepam USP | 19.2 | |
| Mannitol USP (Mannitol-25) | 122.4 | |
| Microcrystalline Cellulose NF (Avicel | 8.0 | |
| PH 101) | | |
| Crospovidone NF (XL-10) | 8.0 | |
| Hydroxypropyl Cellulose NF (Klucel | 2.4 | |
| LF) | | |
| Purified Water (removed during | 113.0 | |
| processing) | | |
| Rapidly Dispersing Microgranules** | | 47.9* |
| Microcrystalline Cellulose NF (Avicel | | 14.0 |
| PH 101) | | |
| Crospovidone NF (XL-10) | | 7.0 |
| Sucralose NF (Splenda) | | 0.495 |
| N-C Natural Peppermint Flavor Code | | 0.607 |
| # 894.037 | | |
| Total (Solids) | 160.0 | 140.0 |

*Based on theoretical potency of 12% by weight temazepam
**95/5 mannitol/Crospovidone XL-10

### Example 3C

### Temazepam Microgranules Mannitol 25 (122.4 kg) and Crospovidone XL-10 (8.0 kg) were co-milled individually by passing the mixture through a Comil® mill. The mannitol, crospovidone, as well as pre-staged microcrystalline cellulose (Avicel PH 101; 8 kg) and crystalline temazepam (Mallinckrodt, 19.2 kg) were blended for about 3-5 minutes. An ODT binder solution was prepared by slowly adding hydroxypropylcellulose (Klucel LF, 2.4 kg) to 113 kg of Purified Water USP in a stainless steel container, with agitation, until dissolved. A Fluid Air FA 300 unit was preheated while empty to reduce the amount of material sticking to the walls of the unit. The pre-blended mixture of mannitol, crospovidone, microcrystalline cellulose, and crystalline temazepam was added to the Fluid Air unit and pre-heated. The aqueous hydroxypropylcellulose solution described above was sprayed onto the blend and granulated. The granulation process was performed in 3 loops with different air flow volumes and filter bag shaking times to minimize the quantity of fines in the resulting granulation. After spraying, the wet granules were dried to reduce the moisture in the granulation to below 2.00%. The dried granules were passed through a 20 mesh market grade screen using a Kason 30″ sifter into fiber drums double lined with one inner anti-static polyethylene bags. The oversized granules were not milled but discarded because of the explosive potential of Temazepam API and granules. Two additional batches (each 160 kg) of granules were also prepared as described above. The process produced temazepam microgranules with very uniform particle size distributions and very high yields which ranged from 96.0% to 98.8%, with the quantity of oversized material ranging from 0.1% to 0.9%. The temazepam assay values ranged from 11.8% to 12.1%. These results show reduced levels of sticking and fines, and no scoring was observed on any of the tablets blended with microcrystalline cellulose (see Table 6 for details on the composition of ODT formulations), NF. All other tablet properties were similar to those that were observed earlier (see Table 7 for tableting properties for the ODT formulations (batch size: 6 kg)). The individual dissolution values at 30 minutes for these 7.5, 15, 22.5 and 30-mg strength tablets were in the range of 98-101%.

TABLE 6
Compositions of Small Scale Reformulated Blends

| | Small Scale Blends | | Scale Up Batches in |
|---|---|---|---|
| | with the New Formula | | the 10 cu.ft. V-Blender |
| | Batch Size = 6.0 kg | | Batch Size = 150.0 kg |
| | 1169-VSP-082 & | 1169-VSP-107, | 1136-JMC-189 & |
| Ingredients | 090 | 115, 122 & 130 | 1236-JMC-011 |
| Temazepam Granulation | 40.00% | 40.00% | 50.00% |
| Mannitol Granulation | 44.18% | 44.22% | 34.22% |
| Microcrystalline cellulose (PH101) | 10.00% | 10.00% | 10.00% |
| Crospovidone, NF | 5.00% | 5.00% | 5.00% |
| Sucralose, NF | 0.35% | 0.35% | 0.35% |
| Nor-Cap Natural Peppermint Flavor | 0.43% | 0.43% | 0.43% |
| FD&C Blue #1, Aluminum Lake | 0.042% | | |
| Totals | 100.00% | 100.00% | 100.00% |

TABLE 7
Tableting Properties of the Reformulated ODT Tablet

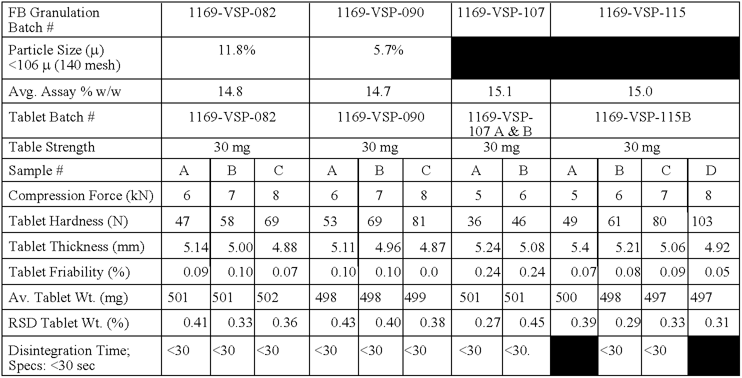

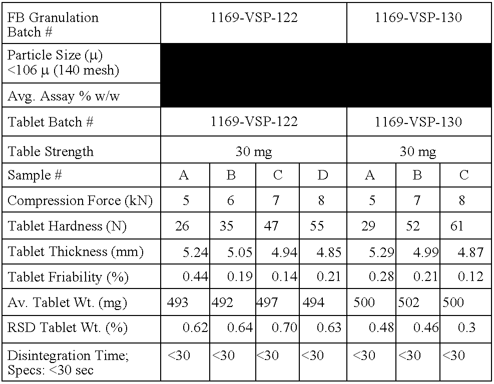

Note:
Shaded areas were not tested.

TABLE 8
Tableting Properties of the Reformulated ODT Tablet

| | FB Granulation Batch # | | | | | | | | | |
|---|---|---|---|---|---|---|---|---|---|---|
| | 1136-JMC-184 | | | | | | 1236-JMC-006 | | | |
| | Tablet Batch # | | | | | | | | | |
| | 1136-JMC-189 | | | | | | 1236-JMC-011 | | | |
| | Tablet Strength | | | | | | | | | |
| | 30 mg | | | | | | 30 mg | | | |
| | Sample # | | | | | | | | | |
| | A | B | C | D | E | F | A | B | C | D |
| Compression Force (kN) | 4.5 | 4.5 | 5 | 5 | 5.5 | 5.5 | 5 | 5.4 | 6 | 6.6 |
| Tablet Hardness (N) | 23 | 25 | 33 | 28 | 36 | 37 | 27 | 30 | 35 | 43 |
| Tablet Thickness (mm) | 5.33 | 5.42 | 5.24 | 5.22 | 5.14 | 5.15 | 5.25 | 5.22 | 5.11 | 5.05 |
| Tablet Friability (%) | 0.45 | 0.71 | 0.25 | 0.24 | 0.08 | 0.17 | 1.14 | NT | 0.4 | 0.31 |
| Av. Tablet Wt. (mg) | 499 | 506 | 498 | 497 | 500 | 500 | 494 | 495 | 496 | 499 |
| RSD Tablet Wt. (%) | 0.55 | 0.75 | 0.80 | 0.73 | 1.14 | 0.68 | 0.33 | 0.49 | 0.34 | 0.36 |

The process was scaled up using 1.5% hydroxypropyl cellulose NF (Klucel LF) during granulation, to provide temazepam granules containing a 12.0 w/w % concentration of temazepam. The two batches (see Table 4/5 for composition of the granulation) were manufactured using a Fluid Air granulator, as follows: The temazepam microgranules in were blended with rapidly dispersing microgranules and other excipients (sucralose, cherry or peppermint flavor, crospovidone XL-10 and microcrystalline cellulose) including microcrystalline cellulose to make final compression blends at a potency of 6.0% by weight temazepam (see Table 5 for composition of the ODT blend). These blends were then compressed into ODTs using a Hata press equipped with the Matsui ExLub System (see Table 8 for tableting properties). The external lubricant (magnesium stearate) spray rates were varied from 1.80 to 3.17 g/min to determine the effect of lubricant spray rate on the tablet dissolution.

The dissolution data showed that neither the ODT binder added during temazepam granulation nor the externally applied lubricant had any effect on the dissolution of resulting tablets (amount of temazepam dissolved was greater than 99% for all formulations at 30 minutes). In addition, no flow related problems were encountered during either short or long compression runs. The above studies confirmed the resolution of the technical issues observed previously during temazepam granulation (without an ODT binder polymer) and tableting of temazepam ODT tablets comprising such granules.

### Example 3D

### Temazepam ODT Compression Blend

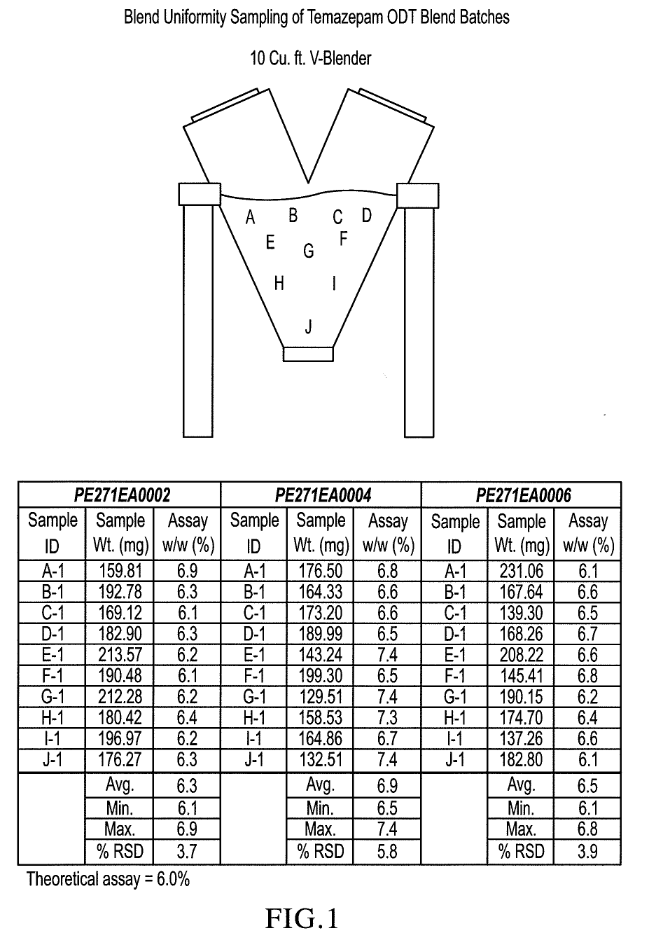

The compression blending of the compositions (Table 5) prepared as described above comprises two blending steps: 1) pre-blending sucralose, peppermint flavor, Crospovidone XL-10, and microcrystalline cellulose in a 2 cu-ft V-blender, and 2) final blending of the pre-blended mixture with rapidly dispersing microgranules of Example 1A and temazepam microgranules of Example 3C (Table 4) in a V-blender. In the pre-blending step, the microcrystalline cellulose, crospovidone, Nor-Cap Natural Peppermint Flavor, and sucralose were first blended in a 2 cu-ft V-blender and then passed through a Comil® mill to break up any clumps and to ensure that sucralose and peppermint flavor were adequately mixed with the other excipients. The peppermint flavor was passed thorough a 40 mesh standard hand screen to break up any lumps before blending with the other excipients. This blended material was then placed inside of the 10 cu-ft V-blender along with the temazepam microgranules prepared as described above (Example 3C) and rapidly dispersing microgranules (Example 1A) and further blended before discharging into fiber drums double lined with anti-static bags. To verify the homogeneity of the final blend a unit dose blending study was performed on the three out of the six blending batches, and samples were collected from 10 different locations for temazepam assays (see FIG. 1 for sampling locations and assay values).

### Example 3E

### Temazepam ODT Compression

Compression blend batches were compressed on a Hata Tablet Press equipped with an Exlub external lubricating system. The starting operating parameters are given in Table 8, and were varied as needed to maintain tablet weight, hardness, thickness and friability within commercial tolerances. The weight range for the tablets was typically maintained with ±4% of the target tablet weight. The ExLub system was started to ensure that the lubricant was spraying properly when the tablet press was running. The tableting parameters, such as fill depth (mm), pre-compression position (mm or kN) and main compression position (mm or kN) were adjusted on the press in order to produce 7.5 mg tablets that meet the specifications shown in Table 9. Following successful set-up, the press was run in ‘Automatic Mode’ until completion of the compression run. During the run, tablets were sampled periodically to ensure that the tablets produced would meet the specifications. One blend batch (batch size: 140 kg of Example 3D) was compressed into 7.5 mg and 30 mg tablets while another blend batch was compressed into 15 mg and 30 mg tablets at a turret speed of 15 RPM. Each batch ran for approximately 6 to 8 hours depending upon the batch size. The tablet weight, hardness and thickness were measured on a sample of five tablets every 30 minutes. Every 60 minutes a sufficient sample was also taken for friability testing. No operating parameters required adjustments during any of the runs to keep the tablet attributes within the specifications given in Table 9, below.

TABLE 9
Operating Parameters for Temazepam ODTs

| Strength/ | | | | |
|---|---|---|---|---|
| Parameter | 7.5 mg Tablets | 15 mg Tablets | 22.5 mg Tablets | 30 mg Tablets |
| Turn Table | 25 | 25 | 25 | 25 |
| Speed | (15-30) | (15-30) | (15-30) | (15-30) |
| Fill Depth | 5.68 | 6.92 | 9.81 | 9.60 |
| | (4.83-6.53) | (5.88-7.96) | (7.85-11.77) | (8.16-11.04) |
| Main Position | 1.71 | 2.39 | 3.15 | 3.46 |
| | (1.54-1.88) | (2.15-2.63) | (2.83-3.46) | (3.11-3.98) |
| Pre-Position | 2.25 | 2.96 | 3.66 | 4.01 |
| | (2.03-2.48) | (2.66-3.26) | (3.29-4.03) | (3.61-4.41) |
| Scale on Feed | 0 | 0 | 0 | 0 |
| Shoe | (0-5) | (0-5) | (0-5) | (0-5) |
| Supply Hopper | 18.0 | 18.0 | 18.0 | 18.0 |
| | (10.0-35.0) | (10.0-35.0) | (10.0-35.0) | (10.0-35.0) |

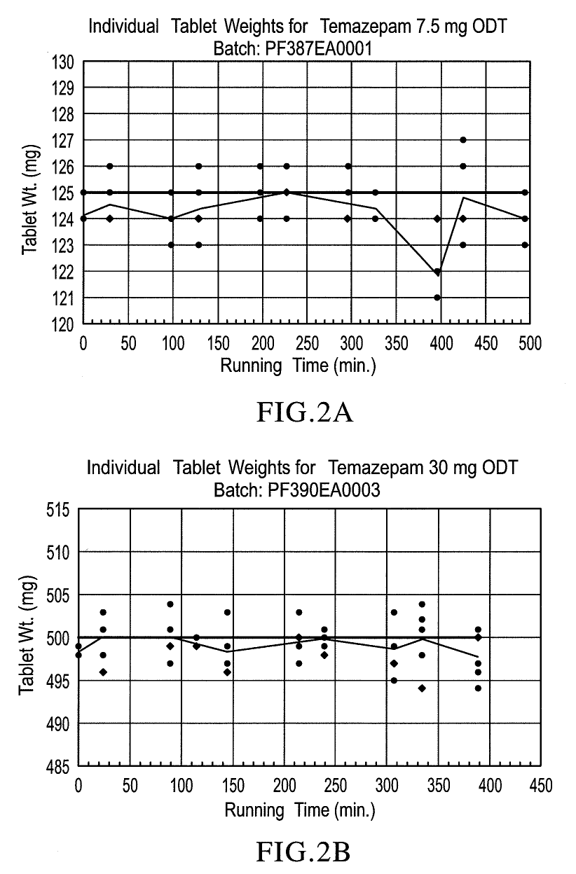

FIG. 2 shows the weight variations for 7.5 and 30 mg temazepam ODTs, throughout each run. The tablet weights in all 12 tableting runs (batches) held very close to the respective targets. The ranges for the mean tablet weight, thickness, hardness and friability as well as the mean relative standard deviation (RSD) are shown in Table 10. No flow-related processing problems or scoring was observed during these tableting runs. In addition, the added binder did not reduce dissolution rates of the temazepam (Table 10) compared to ODTs prepared without the ODT binder (i.e., Examples 1 & 2).

TABLE 10
Properties of Registration Stability Batches of Temazepam ODTs

| | | Tablet | Tablet | Friability | |
|---|---|---|---|---|---|
| | Tablet Weights | Thickness | Hardness | Average (%) | Average |
| ODT | Average (mg) | Average (mm) | Average (N) | (Target: | Compression |
| Strength | RSD (%) | RSD (%) | RSD (%) | NMT 0.6%) | Force (kN) |
| 7.5 mg | 124.2-124.9 | 3.09-3.11 | 27.6-31.7 | 0.19-0.27 | 4.03-4.17 |
| | 0.83-0.99 | 0.35-0.48 | 7.39-8.71 | | |
| 15 mg | 247.4-249.1 | 4.00-4.01 | 5.7-10.0 | 0.18-0.23 | 3.64-4.01 |
| | 0.33-0.45 | 0.41-0.43 | | | |
| 22.5 mg | 372.1-373.2 | 4.92-4.95 | 26.0-26.9 | 0.18-0.28 | 4.12-4.20 |
| | 0.55-0.69 | 0.26-0.30 | 4.85-8.19 | | |
| 30 mg | 499.2-499.7 | 5.15-5.23 | 37.3-40.7 | 0.17-0.23 | 5.26-5.85 |
| | 0.36-0.49 | 0.33-0.45 | 6.77-8.77 | | |

### Example 4

### Example 4A-Study Protocol

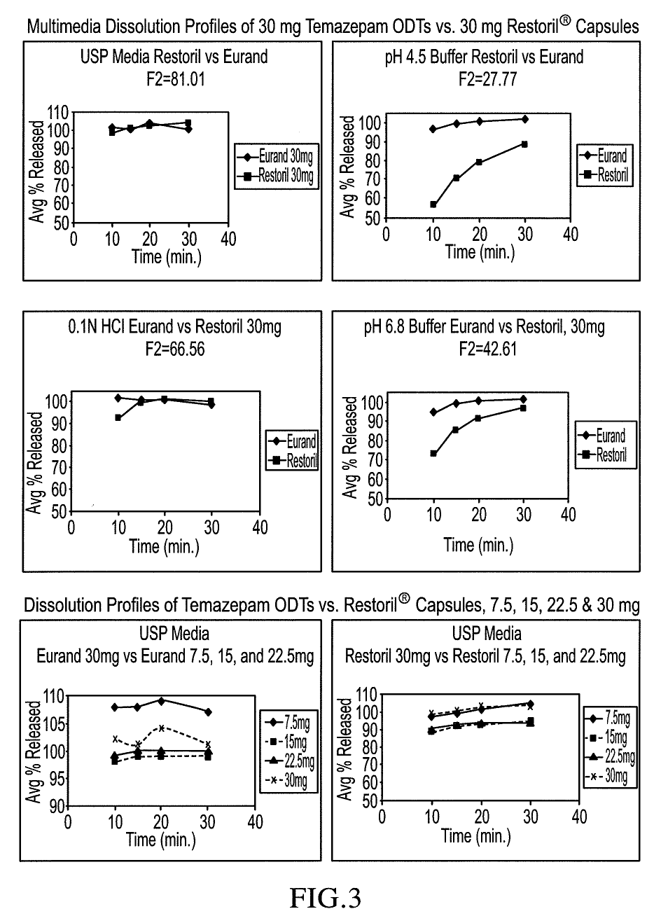

The dissolution profiles for 7.5, 15, 22.5, or 30 mg Temazepam ODTs prepared in Example 3E and Restoril® Capsules are presented in Table 11, below, as well as in FIG. 3

TABLE 11
Dissolution of Temazepam ODTs and Restoril Capsules:
Comparative Data

| | | Cumulative % dissolved | | | | |
|---|---|---|---|---|---|---|
| | | 10 | | 20 | | Range at |
| Lot# | Dose | min | 15 min | min | 30 min | 15 min |

Orally Disintegrating Tablets

| | | | | | | | |
|---|---|---|---|---|---|---|---|
| PF390EA0003 | 30 | mg | 102 | 101 | 104 | 101 | 100-103 |
| PF389EA0003 | 22.5 | | 99 | 100 | 100 | 100 | 98-101 |
| PF388EA0003 | 15 | mg | 98 | 99 | 99 | 99 | 96-100 |
| PF387EA0002 | 7.5 | mg | 108 | 108 | 109 | 107 | 103-114 |

Restoril ® Capsules

| | | | | | | | |
|---|---|---|---|---|---|---|---|
| 9917J50709 | 30 | mg | 99 | 101 | 103 | 104 | 97-106 |
| 9914K53769 | 22.5 | mg | 91 | 93 | 94 | 94 | 89-99 |
| 9916J50044 | 15 | mg | 89 | 92 | 93 | 95 | 87-96 |
| 9915K54294 | 7.5 | mg | 98 | 100 | 102 | 106 | 96-105 |

Two clinical studies were carried out on normal human subjects under fasting and fed conditions. The first clinical study was an open-label, randomized, three-period, crossover study for evaluating the bioequivalence of 30 mg temazepam ODTs taken with and without water, compared to 30 mg temazepam capsules (the reference drug product, Restoril®) in normal human subjects under fasting conditions. The second clinical study was a two-period, crossover study under fed conditions. Both studies used temazepam ODTs (batch size: 570,000 tablets, assay: 102.3%; content uniformity (mean: 99.7%; CV: 1.0%)) and Restoril® (capsule lot #9917J50709 (batch size: 1.9 million units; assay: 99.2%; CU (mean: 100.7%; CV: 1.5%)).

In the fasting study, 54 healthy subjects (47 completed) received three separate drug administrations in three assigned periods, one single 30 mg dose per period. The drug was administered after a 10 hour fast at time 0, Day 1 of the study. The drug administered was either: the temazepam ODT formulation with 240 mL (8 fluid ounces) of room temperature tap water immediately following oral tablet disintegration, the temazepam ODT without water, or a Restoril® Capsule with 240 mL (8 fluid ounces) of room temperature tap water. Food was withheld for a further 4 hours post-dose.

In the fed study, 50 healthy subjects received one 30 mg dose of Temazepam ODT administered with 240 mL (8 fluid ounces) of room temperature tap water immediately following oral tablet disintegration, or a single 30 mg capsule of Restoril® with 240 mL (8 fluid ounces) of room temperature tap water following a 10-hour overnight fast and consumption of a high-fat standardized breakfast.

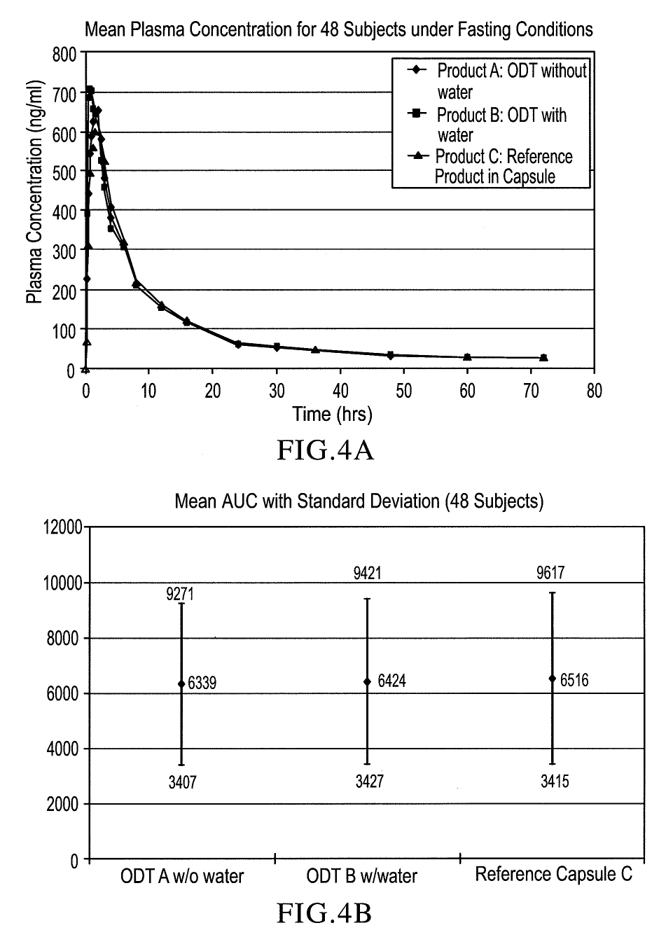

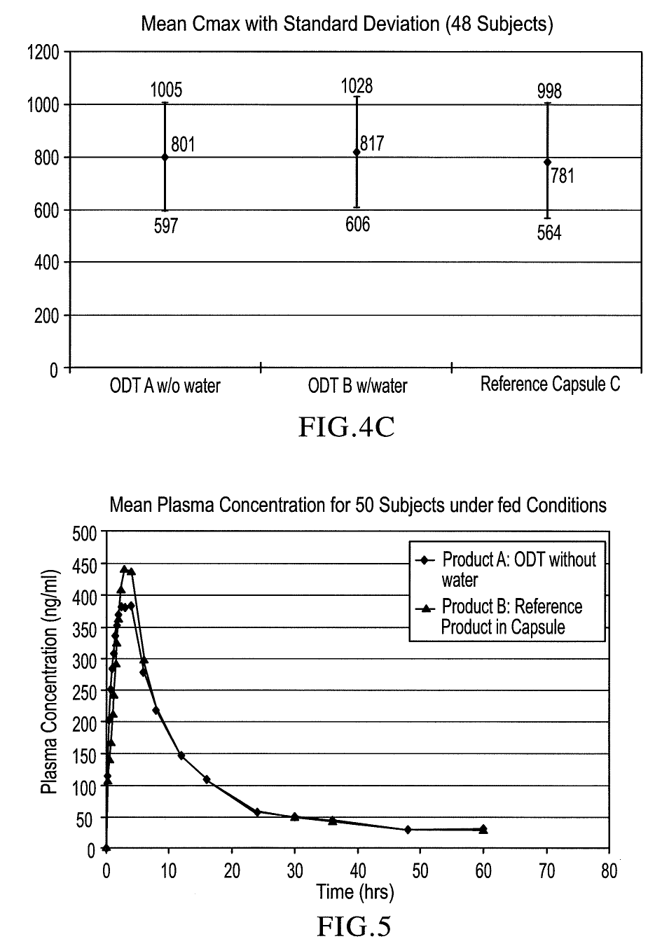

The clinical safety of temazepam was monitored using the following parameters: adverse events, vital signs (sitting blood pressure, pulse, and respiratory rate), health status inquiry, physical examinations, medical history, clinical laboratory values (hematology, chemistry, urinalysis), and impaired judgment evaluations. In each study period, 6 mL blood samples were obtained prior to dosing (up to 1 hour prior to dosing), and at 0.25, 0.50, 0.75, 1, 1.25, 1.50, 1.75, 2, 2.50, 3, 4, 6, 8, 12, 16, 24, 30, 36, 48, 60, and 72 hours post-dose. Plasma was separated from all blood samples and analyzed for temazepam using a validated LC-MS/MS analytical method (calibration range: 20-1200 ng/mL). Clinical safety was monitored by adverse events, vital signs, health status inquiry, physical examination, clinical laboratory values, and impaired judgment evaluations. Parametric general linear model methodology (i.e., non-compartmental method) was used in the analysis of variance of the pharmacokinetic (PK) parameters. From the ANOVA results on log-transformed data, 90% confidence intervals, and intra-subject coefficients of variation were calculated for the AUC_{0-inf} (ng*hr/mL) and Cₘₐₓ (ng/in L). The results obtained in the fasting and fed studies are presented in Table 12 and 13, respectively, while the plasma concentration-time profiles, mean AUC and Cₘₐₓ and their standard deviations are presented in FIGS. 4 and 5. In addition, ANOVA results on the untransformed parameters Tₘₐₓ (time to maximum plasma level, hours), t_{1/2}, and kₑ₁ were obtained. Based on these data, the 30 mg temazepam ODT dosage form, administered with or without water, was found to be bioequivalent to the 30 mg Restoril® capsules dosed with water in the fasted or fed state. Both 30 mg dosage forms of temazepain were found to be safe and well tolerated.

TABLE 12
1 PK Parameters for Temazepam ODT vs Restoril ® Capsule under fasting conditions

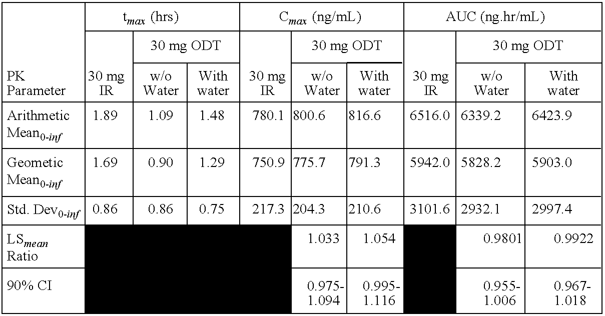

TABLE 13
PK Data for Temazepam ODT vs Restoril Capsule under fed conditions

| | | Geometric LS | Geometric LS | Ratio of Test/Ref |
|---|---|---|---|---|
| PK Parameter | Comparison | mean for ODT | mean for Ref. | (90% CI) |
| AUC0-inf | ODT w/o water | 4716.8 ± 2471.6 | 4874.4 ± 2342.5 | 0.969 |
| (ng * hr/mL) | vs Restoril | | | (0.941-0.997) |
| Cmax (ng/mL) | ODT w/o water | 404.3 ± 142.7 | 500.5 ± 151.1 | 0.810 |
| | vs Restoril | | | (0.753-0.871) |

### Example 5

### Example 5A

### Temazepam Microcaps at 6% Coating

A 5-gallon coacervation tank equipped with an agitator blade 6″+4″ high shear, 3-blade insert, is charged with 10 kg of cyclohexane, and 850 g temazepam crystalline material, 150 g ethylcellulose (Ethocel Standard 100 Premium), 100 g polyethylene (Epoline C-10) is charged into the tank. The tank is heated to approximately 80° C. while stirring at about 200 rpm to dissolve the ethylcellulose. Thereafter, the tank is subjected to controlled-cooling to induce phase separation. Upon cooling to ambient temperature, the microcapsules are filtered, rinsed with fresh cyclohexane, and filtered material is transferred to a fume hood to allow residual cyclohexane to evaporate, thereby providing microencapsulated temazepam with a 6 wt. % ethylcellulose coating.

### Example 5B

### Temazepam ODTs

Sucralose (0.35%), cherry flavor 0.50%), microcrystalline cellulose (Ceolus® KG-1000 from Asahi Kasei, Tokyo, Japan; 5%) and Crospovidone (4.15%) are pre-blended in a 0.25 cu-ft V-blender and further blended with the temazepam taste-masked microparticles (40%) prepared as described above, and rapidly-dispersing microgranules (50%), and compressed into 7.5, 15, 22.5, and 30 mg temazepam ODTs.

We claim:

1. An orally disintegrating tablet composition comprising:
a therapeutically effective amount of at least one drug;
0.5-3% of an ODT binder polymer;
a sugar alcohol and/or saccharide; and
a disintegrant.

2. The orally disintegrating tablet composition of claim 1, wherein the at least one drug is selected from the group consisting of benzodiazepines, analgesics, antihypertensives, antianxiety agents, anticlotting agents, anticonvulsants, anti-diabetic agents, blood glucose-lowering agents, decongestants, antihistamines, anti-inflammatory agents, antitussives, antineoplastics, beta blockers, anti-rheumatic agents, anti-inflammatories, antipsychotic agents, cognitive enhancers, anti-atherosclerotic agents, antiobesity agents, anti-impotence agents, anti-infective agents, anti-infective agents, hypnotic agents, anti-Parkinsonism agents, anti-Alzheimer's disease agents, anti-depressants, and antiviral agents, glycogen phosphorylase inhibitors, cholesterol ester transfer protein inhibitors, CNS (central nervous system) stimulants, dopamine receptor agonists, anti-emetics, gastrointestinal agents, psychotherapeutic agents, opioid agonists, opioid antagonists, anti-epileptic drugs, histamine H₂ antagonists, anti-asthmatic agents, smooth muscle relaxants, and skeletal muscle relaxants.

3. The orally disintegrating tablet composition of claim 1, wherein the at least one drug is temazepam.

4. The orally disintegrating tablet composition of claim 1, prepared by granulating at least one drug, the sugar alcohol and/or saccharide, and the disintegrant in the presence of the ODT binder polymer.

5. The orally disintegrating tablet composition of claim 1, wherein the composition comprises drug particles and rapidly-dispersing microgranules;
the rapidly-dispersing microgranules comprise the sugar alcohol and/or saccharide in combination with the disintegrant; and
the drug particles comprise at least one drug, a sugar alcohol and/or saccharide, and the ODT binder polymer.

6. The orally disintegrating tablet composition of claim 5, wherein the composition substantially disintegrates within about 30 seconds after contact with saliva in the oral cavity or when tested by the <USP 701> Disintegration Test.

7. The orally disintegrating tablet composition of claim 5, wherein the composition substantially disintegrates within about 60 seconds after contact with saliva in the oral cavity or when tested by the <USP 701> Disintegration Test.

8. The orally disintegrating tablet composition of claim 5, wherein the composition releases about 70% or more of the temazepam in 30 minutes when tested for dissolution using US Pharmacopeia Apparatus 2 (paddles at 75 rpm in 900 mL of sodium acetate buffer (pH 4.0) with 0.05% polysorbate).

9. The orally disintegrating tablet composition of claim 5, wherein the rapidly-dispersing microgranules have a mean particle size of about 100-400 μm.

10. The orally disintegrating tablet composition of claim 6, wherein the mean particle sizes of the sugar alcohol and/or saccharide and disintegrant of the rapidly-dispersing granules are each independently about 1-30 μm.

11. The orally disintegrating tablet composition of claim 5, wherein the amount of disintegrant in the rapidly-dispersing microgranules is about 1-10%.

12. The orally disintegrating tablet composition of claim 11, wherein the disintegrant is selected from the group consisting of crospovidone, sodium starch glycolate, cross-linked sodium carboxymethylcellulose, low-substituted hydroxypropylcellulose, and mixtures thereof.

13. The orally disintegrating tablet composition of claim 11, wherein the sugar alcohol is selected from the group consisting of mannitol, alkyl xylitol, sorbitol, maltol, maltitol, and mixtures thereof, and the saccharide is selected from the group consisting of lactose, sucrose, maltose, and mixtures thereof.

14. The orally disintegrating tablet composition of claim 11, wherein the rapidly dispersing microgranules comprise mannitol and crospovidone.

15. The orally disintegrating tablet composition of claim 5, wherein the amount of rapidly-dispersing microgranules is about 50-90% of the total weight of the orally disintegrating tablet composition.

16. The orally disintegrating tablet composition of claim 5, wherein the drug particles have a mean particle size of about 100-400 μm.

17. The orally disintegrating tablet composition of claim 16, wherein the drug particles comprise crystallites of the drug having a mean particle size of about 1-200 μm.

18. The orally disintegrating tablet composition of claim 10, further comprising one or more pharmaceutically acceptable excipients, wherein the drug particles have a mean particle size of about 100-400 μm, the drug particles comprise crystallites of the drug having a mean particle size of about 1-50 μm, and the pharmaceutically acceptable excipients each have a mean particle size of about 1-30 μm.

19. The orally disintegrating tablet composition of claim 5, further comprising a taste-masking layer coating the drug particles.

20. The orally disintegrating tablet composition of claim 19, wherein the taste-masking layer is about 3-10% by weight of the total weight of the temazepam particles.

21. The orally disintegrating tablet composition of claim 19, wherein the taste-masking layer comprises a water insoluble polymer and an optional pore former.

22. The orally disintegrating tablet composition of claim 21, wherein the taste-masking layer comprises a water insoluble polymer and a pore former, and the pore former is about 10-50% of the total weight of the taste-masking layer.

23. The orally disintegrating tablet composition of claim 20, wherein the water insoluble polymer is ethyl cellulose having a viscosity of about 7-100 cps.

24. A method of preparing the composition of claim 1, comprising:
mixing at least one drug, 0.5-3% of an ODT binder polymer, a sugar alcohol and/or saccharide, and a disintegrant; and
compressing said mixture, thereby an forming an orally disintegrating tablet.

25. The method of claim 24, wherein said mixing comprises:
granulating a drug mixture comprising at least one drug, a sugar alcohol and/or saccharide, and the ODT binder polymer, thereby forming drug microgranules;
granulating a disintegrant mixture comprising a sugar alcohol and/or saccharide in combination with a disintegrant, thereby forming rapidly-dispersing microgranules; and
blending the drug microgranules and rapidly-dispersing microgranules.

26. The method of claim 25, further comprising blending the drug microgranules and rapidly-dispersing microgranules with additional pharmaceutically acceptable excipients.

27. The method of claim 25, further comprising coating the drug microgranules with a taste-masking layer.

28. The method of claim 24, wherein said compressing is carried out with a tablet press having an external lubrication system whereby the dies and punches of the tablet press are pre-lubricated.

29. The method of claim 24, wherein said compressing is carried out with a rotary tablet press.

30. A method of treating a patient with a disease or condition comprising administering an effective amount of the orally disintegrating tablet composition of claim 2.

31. The method of claim 30, wherein the disease or condition is a sleeping disorder, and the at least one drug is temazepam.

32. The method of claim 31, wherein said patient with a sleeping disorder has dysphagia.
